(19) United States
(12) Patent Application Publication
(10) Pub. No.: US 2014/0193872 A1
(43) Pub. Date: Jul. 10, 2014

(54) Process for the Conversion of Cellulosic Feedstock Materials

(71) Applicants: Bo Ava Chen, San Diego, CA (US); Ian Dobson, London (GB); Russell F. Heissner, Hingham, MA (US)

(72) Inventors: Bo Ava Chen, San Diego, CA (US); Ian Dobson, London (GB); Russell F. Heissner, Hingham, MA (US)

(21) Appl. No.: 14/105,742

(22) Filed: Dec. 13, 2013

Related U.S. Application Data
(60) Provisional application No. 61/737,565, filed on Dec. 14, 2012.
(60) Provisional application No. 61/737,558, filed on Dec. 14, 2012.

(51) Int. Cl. C12P 7/14

(52) U.S. Cl. CPC C12P 7/14
USPC 435/162

(57) ABSTRACT

A process for converting a cellulosic feedstock to chemical compound, such as an alcohol, or mixture of chemical compounds, wherein one or more process streams can be used in one or more of the other process steps.

This application claims the benefit of U.S. Provisional Patent Application Ser. No. 61/737,565 filed on Dec. 14, 2012, and U.S. Provisional Patent Application Ser. No. 61/737,558 filed on Dec. 14, 2012 and titled “A Sequential Fermentation of Hydrolysate and Solids From a Dilute Acid Hydrolysis of Biomass to Produce Fermentation Products” which are hereby incorporated herein by reference in its entirety.

### BACKGROUND OF THE INVENTION

1. Technical Field

The invention is directed to a process suitable for the conversion of feedstock materials, such as cellulosic feedstock materials, into one or more useful compounds such as glucose or one or more other sugars. It is also directed to the conversion of such sugars into fermentation products such as alcohols. These alcohols, such as ethanol, can be used, for example, as fuels for internal combustion or other engines, as solvents, and as chemical building blocks for the manufacture of other chemicals and polymeric materials.

2. Discussion of Related Art

A desire to produce fuels and chemical building blocks from renewable sources, as well as the desire to reduce the introduction of greenhouse gases such as carbon dioxide in the atmosphere by, for example, the combustion of fossil fuels, have led to the development of process technologies that use gaseous carbon dioxide from the atmosphere to produce fuels and chemical building blocks. A fuel having carbon atoms and where the carbon atoms came from a feedstock such as a cellulosic plant material, will return the carbon in the form of carbon dioxide to the pool of carbon dioxide in the atmosphere when that fuel is combusted in, for example, an eternal combustion engine of an automobile, truck, locomotive, or aircraft. The growth of new plants as cellulosic feedstock materials can use carbon dioxide from that pool thereby completing a cycle of feedstock growth, conversion to fuel, fuel combustion, and re-growth of cellulosic feedstock material without increasing the amount of carbon dioxide released to the atmosphere.

Although various processes have been developed to convert cellulosic feedstock materials into fuels and chemicals, the need exists to improve these processes and to create other improved processes. This invention provides such new and improved processes.

### SUMMARY

In one aspect, this invention provides a process for converting a cellulosic feedstock to a chemical compound, such as an alcohol, or mixture of chemical compounds, comprising processing the feedstock to form a first juice and a first solids comprising cellulose and hemicellulose; hydrolyzing at least part of the first solids to form a first liquid comprising one or more sugars and second solids comprising cellulose; separating in one or more separation apparatuses first liquid from second solids to form a first liquid portion comprising one or more sugars and a solids portion comprising cellulose; fermenting at least part of the one or more sugars in the first liquid portion to form a first fermentation mixture comprising a chemical compound, such as an alcohol, or mixture of chemical compounds; saccharifying in the presence of at least part of the first fermentation mixture at least part of the solids portion to form a second mixture comprising one or more sugars; fermenting at least part of the sugars in the second mixture to form a second fermentation mixture comprising a chemical compound, such as an alcohol, or mixture of chemical compounds; and at least one of the following additional steps of: washing second solids with at least part of the first juice; adding at least a part of the first juice to solids portion exiting the one or more separation apparatuses; combining in a slurry vessel at least part of the first juice with at least a part of the solids portion comprising cellulose; saccharifying solids portion in the presence of at least part of the first juice; and fermenting sugars in the second mixture in the presence of at least part of the first juice.

In another aspect, this invention provides a process for converting a cellulosic feedstock to a chemical compound, such as an alcohol, or mixture of chemical compounds, comprising processing the feedstock to form a first juice comprising one or more sugars and a first solids comprising cellulose and hemicellulose; hydrolyzing at least part of the first solids to form a first liquid comprising one or more sugars and second solids comprising cellulose; separating in one or more separation apparatuses first liquid from second solids to form a first liquid portion comprising one or more sugars and a solids portion comprising cellulose; fermenting at least part of the one or more sugars in the first liquid portion to form a first fermentation mixture comprising a chemical compound, such as an alcohol, or mixture of chemical compounds; saccharifying, optionally in the presence of at least part of the first fermentation mixture, at least part of the solids portion to form a second mixture comprising one or more sugars; fermenting at least part of the sugars in the second mixture to form a second fermentation mixture comprising a chemical compound, such as an alcohol, or mixture of chemical; fermenting at least part of the sugar in the first juice to form a fermented first juice; and at least one of the following additional steps of: washing second solids with at least part of the fermented first juice; adding at least part of the fermented first juice to solids portion exiting the one or more separation apparatuses; combining in a slurry vessel at least part of the fermented first juice with at least part of the solids portion comprising cellulose; saccharifying solids portion in the presence of at least part of the fermented first juice; fermenting sugars in the second mixture in the presence of at least part of the fermented first juice; processing the feedstock with at least part of the fermented first juice; and hydrolyzing at least part of the first solids with at least part of the fermented first juice.

In another aspect, this invention provides a process for converting a cellulosic feedstock to a chemical compound, such as an alcohol, or mixture of chemical compounds, comprising: processing the feedstock to form a first juice and a first solids comprising cellulose and hemicellulose; hydrolyzing at least part of the first solids to form a first liquid comprising one or more sugars and second solids comprising cellulose; separating in one or more separation apparatuses first liquid from second solids to form a first liquid portion comprising one or more sugars and a solids portion comprising cellulose; fermenting at least part of the one or more sugars in the first liquid portion to form a first fermentation mixture comprising a chemical compound, such as an alcohol, or mixture of chemical compounds; saccharifying, optionally in the presence of at least part of the first fermentation mixture, at least part of the solids portion to form a second mixture comprising one or more sugars; fermenting at least part of the sugars in the second mixture to form a second fermentation mixture comprising a chemical compound, such as an alcohol, or mixture of chemical compounds; and at least one of the following additional steps of: washing in the separation apparatus second solids with at least part of the first fermentation mixture; adding at least part of the first fermentation mixture to solids portion exiting the one or more separation apparatuses; combining in a slurry vessel at least part of the first fermentation mixture with at least part of the solids portion comprising cellulose; saccharifying solids portion in the presence of at least part of the first fermentation mixture; fermenting sugars in the second mixture in the presence of at least part of the first fermentation mixture; processing the feedstock with at least part of the first fermentation mixture; and hydrolyzing at least part of the first solids with at least part of the first fermentation mixture.

In another aspect, this invention provides a process for converting a cellulosic feedstock to a chemical compound, such as an alcohol, or mixture of chemical compounds, comprising processing the feedstock to form a first juice and a first solids comprising cellulose and hemicellulose; hydrolyzing at least part of the first solids to form a first liquid comprising one or more sugars and second solids comprising cellulose; separating in one or more separation apparatuses first liquid from second solids to form a first liquid portion comprising one or more sugars and a solids portion comprising cellulose; fermenting at least part of the one or more sugars in the first liquid portion to form a first fermentation mixture comprising a chemical compound, such as an alcohol, or mixture of chemical compounds, saccharifying, optionally in the presence of at least part of the first fermentation mixture, at least part of the solids portion to form a second mixture comprising one or more sugars; fermenting at least part of the sugars in the second mixture to form a second fermentation mixture comprising a chemical compound, such as an alcohol, or mixture of chemical compounds, and at least one of the following additional steps of: washing in the separation apparatus second solids with part of the second fermentation mixture; adding part of the second fermentation mixture to the solids portion exiting the one or more separation apparatuses; combining in a slurry vessel a part of the second fermentation mixture with at least a part of the solids portion comprising cellulose; processing the feedstock with part of the second fermentation mixture; and hydrolyzing at least part of the first solids with part of the second fermentation mixture.

In another aspect, this invention provides a process for converting a cellulosic feedstock to chemical compound, such as an alcohol, or mixture of chemical compounds, comprising hydrolyzing the feedstock to form a first liquid comprising one or more sugars and first solids comprising cellulose; separating in one or more separation apparatuses first liquid from first solids to form a first liquid portion comprising one or more sugars and a solids portion comprising cellulose; fermenting at least part of the one or more sugars in the first liquid portion to form a first fermentation mixture comprising a chemical compound, such as an alcohol, or mixture of chemical compounds, saccharifying at least part of the solids portion to form a second mixture comprising one or more sugars; fermenting at least part of the sugars in the second mixture to form a second fermentation mixture comprising a chemical compound, such as an alcohol, or mixture of chemical compounds, and at least one of the following additional steps of: washing in the separation apparatus first solids with at least part of the first fermentation mixture; adding at least part of the first fermentation mixture to solids portion exiting the one or more separation apparatuses; combining in a slurry vessel at least part of the first fermentation mixture with at least part of the solids portion comprising cellulose; saccharifying solids portion in the presence of at least part of the first fermentation mixture; fermenting sugars in the second mixture in the presence of at least part of the first fermentation mixture; and hydrolyzing at least part of the feedstock with at least part of the first fermentation mixture.

The chemical compound produced by the fermentation of the sugars as set for the above can be, for example, an alcohol such as one or more of ethanol, n-propanol, isopropanol, and n-butanol, 2-butanol, isobutanol or tertiary butanol or a dialcohol such as 2,3-butanediol. Suitably, the alcohol is ethanol or isobutanol, and more suitably, ethanol. The alcohol can be any mixture comprising of any two or more of these alcohols. The chemical compound can be one or more of an alcohol, such as the alcohols mentioned above, ketone, carboxylic acid, aldehyde, ester or hydrocarbon. Representative ketones include, but are not limited to, acetone and methylethylketone. Representative carboxylic acids include, but are not limited to, formic acid, acetic acid, propionic acid, carboxylic acids having four carbon atoms such as n-butyric acid, iso-butyric acid, and 2-butanoic acid, carboxylic acids having five carbon atoms such as methylbutanoic acid isomers and carboxylic acids having six to twenty, or six to eighteen carbon atoms as well as compounds with mixed functionality such as keto-acids. Representative aldehydes include, but are not limited to, acetaldehyde, propionaldehyde, n-butyraldehyde, iso-butyraldehyde, and methylbutanals. Representative esters include, but are not limited to, methyl acetate, ethyl acetate, ethylbutyrate isomers, butyl acetate isomers, butyl butyrate isomers and higher molecular weight esters. Representative hydrocarbons include, but are not limited to, ethylene, but-1-ene, but-2-ene, and isobutylene. In the processes set forth above, the one or more chemical compounds in the first fermentation mixture can be the same as or can be different from the one or more chemical compounds in the second fermentation mixture.

The term “fermenting” means the conversion of the one or more sugars to the one or more other chemical compounds, such as those set for the above, using one or more living organisms or one or more enzymes where the one or more enzymes may or may not be part of one or more living organisms. The living organisms can be, for example, yeasts, bacteria, other fungi, archaea, algae, and the like. One or more yeasts are particularly advantageous in the process of this invention.

Additional features and advantages of the invention will be set forth in the description that follows, being apparent from the description or learned by practice of embodiments of the invention.

It is to be understood that both the foregoing summary and the following detailed description and drawings are exemplary and explanatory, and are intended to provide further explanation of the invention as claimed.

### BRIEF DESCRIPTION OF THE DRAWINGS

FIG. 1 is a process flow diagram showing a process for the conversion of a cellulosic feedstock to ethanol in accordance with an embodiment of this invention.

FIG. 2 is a process flow diagram showing a process for the conversion of a cellulosic feedstock to ethanol in accordance with embodiments of this invention where a first juice stream and a fermented fist juice are used in various process steps.

FIG. 3 is a process flow diagram showing a process for the conversion of a cellulosic feedstock to ethanol in accordance with embodiments of this invention where the hydrolyzate from a cellulosic feedstock is used in various process steps.

FIG. 4 is a process flow diagram showing a process for the conversion of a cellulosic feedstock to ethanol in accordance with embodiments of this invention where a first fermentation mixture and a second fermentation mixture are used in various process steps.

FIG. 5 is a process flow diagram showing a process for the conversion of a cellulosic feedstock to ethanol in accordance with embodiments of this invention.

FIG. 6 is a process flow diagram showing a process for the conversion of a cellulosic feedstock to ethanol in accordance with embodiments of this invention where a first juice stream and a fermented fist juice are used in various process steps.

FIG. 7 is a process flow diagram showing a process for the conversion of a cellulosic feedstock to ethanol in accordance with embodiments of this invention where the hydrolyzate from a cellulosic feedstock is used in various process steps.

FIG. 8 is a process flow diagram showing a process for the conversion of a cellulosic feedstock to ethanol in accordance with an embodiment of this invention where a second fermentation mixture is used in various process steps.

### DETAILED DESCRIPTION

Although the following description of embodiments of the invention comprises the production of ethanol from cellulosic materials, such as lignocellulosic materials, it is to be understood that the sugars and sugar oligomers that are produced in the described embodiments can be used to prepare other chemical compounds such as one or more alcohols, such as, for example, one or more butanols, by chemical transformations accomplished, for example, by enzymatic processes and/or by one or more microorganisms.

In one embodiment, this invention relates to a process for the production of ethanol and intermediate sugars that comprises, as the main steps, processing a cellulosic feedstock including obtaining a sugar solution, or also referred to herein as type of “first juice,” from the feedstock; hydrolysis of the remaining feedstock material to produce solids comprising a cellulosic component, separation of the solids from liquid produced by the hydrolysis to form a solids portion and a liquid hydrosylate, detoxification of the hydrosylate, conversion, for example, by a fermentation, of the primarily five carbon (C5) sugars in the detoxified hydrolyzate to form a first fermentation mixture comprising ethanol, saccharification of the solids portion, optionally in the presence of at least a portion of the first fermentation mixture, whereby cellulose in the cellulosic component of the solids portion is, suitably, enzymatically hydrolyzed to form an aqueous mixture comprising primarily six carbon (C6) sugars; conversion, for example by fermentation, of C6 sugars in the aqueous mixture to form a second fermentation mixture comprising ethanol, and directing at least part of one or more of the first juice, fermented first juice, the hydrolysate, and the first fermentation mixture to one or more of the steps of processing the feedstock, hydrolysis of the feedstock, the separation of the solids from the liquid produced by the hydrolysis, and the saccharification the solids, to thereby improve the process of manufacturing ethanol from a cellulosic feedstock. The saccharification of the solids portion and the fermentation of the primarily C6 sugars formed by the saccharification can occur simultaneously, i.e., a simultaneous saccharification and fermentation process, or “SSF.” These steps, and additional process steps such as directing at least part of the second fermentation mixture to one or more of the steps of preparing the feedstock, hydrolysis of the feedstock, the separation of the solids from the liquid produced by the hydrolysis, will be described in more detail below.

The phrase “at least part of” when referring to a component, process product, process stream, or the like can mean, one or more of or any one of, at least about 1%, at least about 2%, at least about 5%, at least about 10%, at least about 20%, at least about 30%, at least about 40%, at least about 50%, at least about 60%, at least about 70%, at least about 80%, at least about 90%, at least about 95%, or at least about 99%, or any range bounded by two of the above percentages such as, for example, about 40% to about 80%, and can mean 100%, where, if the component, process product, process stream or the like, is a solid, the % is weight percent and if a liquid, the % is volume percent. The phrase “part of”, i.e., without the modifying words “at least” when referring to a component, process product, process stream, or the like can mean, one or more of or any one of, at least about 1%, at least about 2%, at least about 5%, at least about 10%, at least about 20%, at least about 30%, at least about 40%, at least about 50%, at least about 60%, at least about 70%, at least about 80%, at least about 90%, at least about 95%, or at least about 99%, or any range bounded by two of the above percentages such as, for example, about 40% to about 80%, but not 100%, where, if the component, process product, process stream or the like, is a solid, the % is weight percent and if a liquid, the % is volume percent.

### Feedstock

The term “feedstock” or “cellulosic feedstock,” as used herein, refers to any composition comprising cellulose, optionally, hemicellulose, and optionally lignin. A feedstock that is specifically referred to as lignocellulosic necessarily comprises lignin.

Feedstock which can be hydrolyzed and saccharified according to the processes of this disclosure can include agricultural crops and agricultural waste such as, for example, seeds, grains, corn stalks, corn byproducts, corn stover, corn fiber, corn cobs, and corn husks, grass, bagasse, such as sugar cane bagasse and energy cane bagasse, straw, for example, straw from rice, wheat, buckwheat, amaranth, rye, millet, oat, barley, rape, sorghum and spelt straw. Feedstock which can be hydrolyzed and saccaharified according to the processes of this disclosure can include tubers, for example, beets, such as sugar beets, and potatoes.

The feedstock can also include, without limitation, plant waste or byproducts of food processing or industrial processing such as wood chips, wood bark, wood saw dust, and other wood byproducts, wood waste and wood processing waste, where the wood, chips, bark, sawdust and other wood byproducts, wood waste and wood processing waste can be deciduous or coniferous wood, hardwood or softwood. Feedstock also includes paper and paper byproducts, paper pulp, paper waste, paper mill waste, and recycled paper such as recycled newspaper, recycled printer paper, and the like. Other feedstocks include, without limitation, soybean, rapeseed, barley, rye, oats, wheat, sorghum, sudan, milo, bulgur, rice, forest residue, and agricultural residue.

A lignocellulosic feedstock is suitably a grass and can be plants from the grass family. The proper name is the family known as Poaceae or Gramineae in the class Liliopsida (the monocots) of the flowering plants. Plants of this family are usually called grasses, and include bamboo. There are believed to be about 600 genera and some 9,000-10,000 or more species of grasses (Kew Index of World Grass Species).

Poaceae includes the staple food grains and cereal crops grown around the world, lawn and forage grasses, and bamboo.

Most of the grasses divide into two physiological groups, using the C3 and C4 photosynthetic pathways for carbon fixation. The C4 grasses have a photosynthetic pathway linked to specialized leaf anatomy that particularly adapts them to hot climates and an atmosphere low in carbon dioxide. C3 grasses are referred to as “cool season grasses” while C4 plants are considered “warm season grasses.”

Grasses may be annual or perennial. Examples of annual cool season grasses are wheat, rye, annual bluegrass such as annual meadowgrass, Poa annua and oat. Examples of perennial cool season are orchardgrass, such as cock's foot (Dactylis glomerata), fescue (Festuca spp.), Kentucky bluegrass and perennial ryegrass (Lolium perenne). Examples of annual warm season grasses are corn, sudangrass and pearl millet. Examples of perennial warm season grasses are big bluestem, indiangrass, bermudagrass and switchgrass.

One classification of the grass family recognizes twelve subfamilies, all of which can be feedstock in embodiments of this invention: These are 1) anomochlooideae, a small lineage of broad-leaved grasses that includes two genera (Anomochloa, Streptochaeta); 2) Pharoideae, also known as Poaceae, a small lineage of grasses that includes three genera, including Pharus and Leptaspis; 3) Puelioideae, a small lineage that includes the African genus Puelia; 4) Pooideae, which includes wheat, barley, oats, brome-grass (Bromus) and reed-grasses (Calamagrostis); 5) Bambusoideae, which includes bamboo; 6) Ehrhartoideae, which includes rice, and wild rice; 7) Arundinoideae, which includes the giant reed and common reed 8) Centothecoideae, a small subfamily of 11 genera that is sometimes included in Panicoideae; 9) Chloridoideae, including the lovegrasses (Eragrostis, ca. 350 species, including teff), dropseed grasses (Sporobolus, some 160 species), finger millet (Eleusine coracana (L.) Gaertn.), and the muhly grasses (Muhlenbergia. ca. 175 species); 10) Panicoideae including panic grass, maize, sorghum, sugar cane, most millets, fonio and bluestemn grasses; 11) Micrairoidcae; 12) Danthoniodieae, including pampas grass; with Poa which is a genus of about 500 species of grasses, native to the temperate regions of both hemisphere. Agricultural grasses grown for their edible seeds are called cereals. Three common cereals are rice, wheat and maize (corn). Of all crops, 70% are grasses. Feedstocks includes all of these grasses.

A suitable feedstock is selected from the group consisting of the energy crops. In a further embodiment, the energy crops are grasses. Suitable grasses as feedstocks include Napier Grass or Uganda Grass, such as Pennisetum purpureum; or, Miscanthus; such as Miscanthus giganteus and other varieties of the genus miscanthus, or Indian grass, such as Sorghastrum nutans; or, switchgrass, for example, as Panicum virgatum or other varieties of the genus Panicum, giant reed (arundo donax), energy cane (saccharum spp.). In some embodiments the feedstock is sugarcane, which refers to any species of tall perennial grasses of the genus Saccharum.

Other suitable types of feedstock include quinoa, milo stubble, citrus waste, urban green waste or residue, food manufacturing industry waste or residue, cereal manufacturing waste or residue, hay, grain cleanings, spent brewer's grain, rice hulls, salix, spruce, poplar, eucalyptus, Brassica carinata residue, Antigonum leptopus, sweetgum, Sericea lespedeza, Chinese tallow, hemp, Sorghum bicolor, soybeans and soybean products such as, for example, soybean leaves, soybeans stems, soybean pods, and soybean residue, sunflowers and sunflower products, such as, for example, leaves, sunflower stems, seedless sunflower heads, sunflower hulls, and sunflower residue, Anrundo, nut shells, deciduous leaves, cotton fiber, manure, coastal Bermuda grass, clover, Johnsongrass, flax, amaranth and amaranth products such as, for example, amaranth stems, amaranth leaves, and amaranth residue and alfalfa.

For wood as a feedstock, the feedstock includes hardwood and softwood. Examples of suitable softwood and hardwood trees as a feedstock include, but are not limited to, the following: pine trees, such as loblolly pine, jack pine, Caribbean pine, lodgepole pine, shortleaf pine, slash pine, londuran pine, Masson's pine, Sumatran pine, western white pine, egg-cone pine, longleaf pine, patula pine, maritime pine, ponderosa pine, Monterey pine, red pine, eastern white pine, Scots pine, araucaria tress; fir trees, such as Douglas fir; and hemlock trees, plus hybrids of any of the foregoing. Additional examples include, but are not limited to, the following: eucalyptus trees, such as Dunn's white gum, Tasmanian blue gum, rose gum, Sydney blue gum, Timor white gum, and the E. urograndis hybrid; populus trees, such as eastern cottonwood, bigtooth aspen, quaking aspen, and black cottonwood; and other hardwood trees, such as red alder, Sweetgum, tulip tree, Oregon ash, green ash, and willow, plus hybrids of any of the foregoing.

The feedstock can be one or more of: a miscanthus, for example, Miscanthus floridulus, Miscanthus giganteus, Miscanthus sacchariflorus, Miscanthus sinensis, Miscanthus tinctorius, Miscanthus transmorrisonensis, Erianthus, such as, E. acutecarinatus, E. acutipennis-E. adpressus, E. alopecuroides, E. angulatus, E. angustifolius, E. armatus, E. articulatus, E. arundinaceus, E. asper, E. aureus, E. bakeri, E. balansae, E. beccarii, E. bengalensis, E. biaristatus, E. bifidus, E. birmanicus, E. bolivari, E. brasilianus, E. brevibarbis, E. capensis, E. chrysothrix, E. ciliaris, E. clandestinus, E. coarctatus, E. compactus, E. contortus, E. cumingii, E. cuspidatus, E. decus-sylvae, E. deflorata, E. divaricatus, E. dohmi, E. ecklonii, E. elegans, E. elephantinus, E. erectus, E. fallax, F. fastigiatus, E. filifolius, E. fischerianus, E. flavescens, E. flavipes, E. flavoinflatus, E. floridulus, E. formosanus, E. formosus, E. fruhstorferi, E. fulvus, E. giganteus, E. glabrinodis, E. glaucus, E. griffithii, E. guttatus, E. hexastachyus, E. hookeri, E. hostii, E. humbertianus, E. inhamatus, E. irritans, E. jacquemontii, E. jamaicensis, E. japonicus, E. junceus, E. kajkaiensis, F. kanashiroi, E. lancangensis, E. laxus, E. longesetosus, E. longifolius, E. longisetosus, E. longisetus, E. lugubris, E. luzonicus, E. mackinlayi, E. macratherus, E. malcolmi, E. manueli, E. maximus, E. mishmeensis, E. mollis, E. monsticrii, E. munga, E. munja, E. nepalensis, E. nipponensis, E. nudipes, E. obtusus, E. orientalis, E. pallens, E. parviflorus, E. pedicellaris, E. perrieri, E. pictus, E. pollinioides. E. procerus, E. pungens. E. purpurascens, E. purpureus. E. pyramidalis, E. ravennae, E. rehni, E. repens, E. rockii, E. roxburghii, E. ruflpilus, E. rufus, E. saccharoides, E. sara, E. scriptorius, E. sesquimetralis, E. sikkimensis, E. smallii, E. sorghum, E. speciosus, E. strictus, E. sukhothaiensis, E. sumatranus, E. teretifolius, E. tinctorius, E. tonkinensis, E. tracyi, E. trichophyllus, E. trinii, E. tristachyus, E. velutinus, E. versicolor, E. viguieri, E. villosus, E. violaceus, E. vitalisi, E. vulpinus, E. wardii, E. williamsii; energy cane, such as sugar cane, for example, S. acinaciforme, S. aegyptiacum, S. alopecuroides, S. alopecuroideum, S. alopecuroidum, S. alopecurus, S. angustifolium, S. antillarum, S. appressum, S. arenicola, S. argenteum, S. arundinaceum, S. asperum, S. atrorubens, S. aureum, S. balansae, S. baldwini, S. baldwinii, S. barberi, S. barbicostatum, S. beccarii, S. bengalense, S. benghalense, S. bicome, S. biflorum, S. boga, S. brachypogon, S. bracteatum, S. brasilianum, S. brevibarbe, S. brevifolium, S. brunneum, S. caducum, S. caffrosum, S. canaliculatum, S. capense, S. casi, S. caudatum, S. cayennense, S. chinense, S. ciliare, S. coarctatum, S. confertum, S. conjugatum, S. contortum, S. contractum, S. cotuliferum, S. cylindricum, S. deciduum, S. densum, S. diandrum, S. dissitiflorum, S. distichophyllum, S. dubium, S. ecklonii, S. edule, S. elegans, S. elephantinum, S. erianthoides, S. europaeum, S. exaltatum, S. fallax, S. fasciculatum, S. fastigiatum, S. fatuum, S. filifolium, S. filiforme, S. floridulum, S. formosanum, S. fragile, S. fulvum, S. fuscum, S. giganteum, S. glabrum, S. glaga, S. glaucum, S. glaza, S. grandiflorum, S. griffithii, S. hildebrandtii, S. hirsutum, S. holcoides, S. hookeri, S. hybrid, S. hybridum, S. indum, S. in[beta]rmum, S. insulare, S. irritans, S. jaculatorium, S. jamaicense, S. japonicum, S. juncifolium, S. kajkaiense, S. kanashiroi, S. klagha, S. koenigii, S. laguroides, S. longifolium, S. longisetosum, S. longisetum, S. lota, S. luzonicum, S. macilentum, S. macrantherum, S. maximum, S. mexicanum, S. modhara, S. modhua, S. monandrum, S. moonja, S. munja, S. munroanum, S. muticum, S. narenga, S. nareya, S. negrosense, S. obscurum, S. occidentale, S. officinale, S. officinalis, S. officinarum, S. palisoti, S. pallidum, S. paniceum, S. panicosum, S. pappifenun, S. parviflorum, S. pedicellare, S. perrieri, S. polydactylum, S. polystachyon, S. polystachyum, S. porphyrocomnum, S. praegrande, S. procerum, S. propinquum, S. punctatum, S. purpuratum, S. rara, S. rarum, S. ravennae, S. repens, S. reptans, S. revennae, S. ridleyi, S. robustum, S. roseum, S. rubicundum, S. ru[beta]pilum, S. rufum, S. sagittatum, S. sanguincum, S. sape, S. sara, S. sarpata, S. scindicus, S. semidecumbens, S. seriferum, S. sibiricum, S. sikkimense, S. sinense, S. sisca, S. soltwedeli, S. sorghum, S. speciosissimum, S. sphaclatum, S. spicatum, S. spontaneum, S. spontaneum, S. stenophyllum, S. stewartii, S. strictum, S. teneriffae, S. tenuius, S. ternatum, S. thunbergii, S. tinctorium, S. tridentatum, S. trinii, S. tripsacoides, S. tristachyum, S. velutinum, S. versicolor, S. viguieri, S. villosum, S. violaceum, S. wardii, S. warmingianum, S. williamsii; hybrids, for example, L 99-233, L 99-226, L79-1001, L 79-1002, L 99-233, L 99-226, HoCP 91-552, HoCP 91-555, Ho 00-961, Ho 02-113, Ho 03-19, Ho 03-48, Ho 99-51, Ho 99-58, US 72-114, Ho 02-144, Ho 06-9002; a sorghum, such as, Sorghum almum, Sorghum amplum, Sorghum angustum, Sorghum arundinaceum, Sorghum bicolor, Sorghum bicolor subsp. drummondii—Sudan grass, Sorghum brachypodum, Sorghum bulbosum, Sorghum burmahicum, Sorghum controversum, Sorghum drummondii, Sorghum ecarinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum propinquum, Sorghum purpureosericeum, Sorghum stipoideumn, Sorghum timorense, Sorghum trichocladum, Sorghum versicolor, Sorghum virgatum, Sorghum vulgare, hybrids, such as sugar cane×Miscanthus or sugar cane×Erianthus; Napier grass (elephant grass), for example, Pennisetum purpureum; or switch grass, for example, Panicum virgatum.

The feedstock can be on or more of rice, stover, wheat, maize, maize stover, sorghum, sorghum stover, sweet sorghum, sweet sorghum stover, cotton, cotton remnant, cassava, sugar beet pulp, soybean, rapeseed, jatropha, switchgrass, miscanthus, other grasses, timber, agricultural waste, manure, dung, sewage, municipal solid waste, any other suitable feedstock material, and/or the like.

Feedstocks that contain cellulose and contain one or more sugars such as one or more of sucrose, glucose, and fructose, are particularly suitable for use in embodiments of this invention. The amount of sucrose in the feedstock on a percent dry weight basis can be, for example, at least about 0.1 percent, at least about 1 percent, at least about 5 percent, and can, for example, be up to about 15 percent, up to about 40 percent, or up to about 60 percent. For example, the amount of sucrose in the feedstock can be about 0.1 to about 15 weight percent, about 5 to about 40 weight percent, or about 10 to about 60 weight percent.

The feedstock can be used either in a green state, that is feedstock that is freshly harvested from the farm or plantation where it is grown, or it can be aged and dried or at least partially dried.

### Feedstock Processing

The feedstock is typically processed before it is used in processes to make, for example, alcohols in accordance with embodiments of this invention. For example, such processing the can include comminuting the feedstock, suitably by mechanical means using, for example, one or more commercially available heavy duty shredder machines. The feedstock can be comminuted so that the average particle size of the feedstock is about 0.1 millimeter, or about 1 millimeter or about 5 millimeters. In some cases the feedstock is shredded whole, for example, in the case of a feedstock that is a cane, without removing leaves or other plant matter from the cane. Magnets can be used to remove any extraneous ferrous debris, or other metallic debris that is attracted by a magnet and that may be present in the comminuted feedstock.

Processing the feedstock, whether or not comminuted, suitably includes pressing the feedstock or otherwise treating the feedstock to remove water, if any, contained in the feedstock. One or more suitable apparatus for pressing the water from the feedstock can be used. If the feedstock is in the green state it typically has appreciable amounts of water that can be removed from the feedstock by, for example, pressing. If the feedstock contains a sugar such as one or more of sucrose, glucose, fructose or galactose the water that is removed suitably contains such sugar or sugars. The water that is removed by pressing the feedstock, or other suitable treatment method to remove water, is referred to herein as the first juice. Typically it does contain one or more sugars.

Additional water can be added to the pressing or other process treatment used to remove additional sugar or sugars. For example, the weight ratio of water added to the feedstock during such pressing or other processing treatment to remove water can be about 0.1:1 to about 10:1, or about 1:1 to about 10:1 or about 3:1 to about 5:1. As described in more detail below, such water can be in the form of one or more process streams produced by one or more embodiments of this inventions that contain water. In some feedstock processing suitable for use in the processes disclosed herein, a series of presses, such as roller mills, are used to press out the water containing the sugar or sugars. For example one, or two, or three, or four presses can be arranged in series. For example, a feedstock that contains a sugar, and where, for example, it has been comminuted, is fed to the first press where it is pressed to produce first solids and a first liquid containing sugar. The first solids are fed to a second press where it is pressed to produce second solids and a second liquid containing sugar. The second solids are fed to a third press where it is pressed to produce third solids and a third liquid. Water, in this element of the process, can be added to the solids between the first and second press or at the second press, between the second and third press, or at the third press, or at any or all of these locations. The liquids from the third press can be recycled to the first solids, to the second solids, or to both the first and second solids. The second liquid can be combined with the first liquid to form an aqueous, sugar-containing mixture, as a first juice. The amount of sugar and the type of sugar in the first juice will depend on, for example, the amount of the sugar in the feedstock, the number of stages of pressing, the degree of pressing, the amount of water or other liquid added to assist with the pressing and extraction of the sugar from the feedstock, and other factors. Suitably the sugar comprises, for example, one or more of sucrose, glucose, and fructose. Suitably the press is a roller mill. The amount of sugar in the first juice can be about 0.1 to about 50 weight percent of the first juice, about 5 to about 20 weight percent of the first juice, and, suitably about 5 to about 6 weight percent of the first juice. For example, the amount of sucrose in the first juice can be about 0.1 to about 50 weight percent of the first juice, about 5 to about 20 weight percent of the first juice, and, suitably about 5 to about 6 weight percent of the first juice. The processing of the feedstock by, for example, pressing and optionally adding water or other liquid to the process such as described above, can be conducted so that at least about 75 percent of the water soluble sugars such as one or more of sucrose, glucose, and fructose contained in the feedstock are removed from the feedstock, or at least about 80 percent, or at least about 85 percent, or at least about 90 percent, or at least about 95 percent of such sugars in the feedstock are removed. Suitably all or substantially all of such sugars are removed and are in the first juice.

The feedstock processed as described above results in a solid, that comprises cellulose, optionally hemicellulose and optionally lignin, and a first juice comprising water.

### Feedstock Hydrolysis and Separation

Feedstock that is lignocellulosic, referred to herein as a lignocellulosic feedstock, comprises primarily cellulose, which is recognized to be a polymer of glucose linked by β-1,4-glucosidic bonds, hemicellulose, which is recognized to be a polysaccharide composed of different five-carbon(C5) sugars and six-carbon (C6) sugars linked by variety of different 3 and a linkages, and lignin, which is recognized to be a complex polymer having phenyl propane units linked by ether or carbon-carbon bonds. Feedstocks, such as lignocellulosic feedstocks, can be subjected to a hydrolysis reaction, suitably in the presence of added water, during which at least part of the hemicellulose if present in the feedstock is hydrolyzed to oligomeric and/or monomeric sugars producing a liquid stream containing the sugars and the crystalline structure of cellulose is damaged, facilitating further hydrolysis, for example enzymatic hydrolysis, of the remaining solid cellulose which is typically in the form of fibers. The feedstock for this hydrolysis reaction can be the solids produced by one or more of the feedstock processing steps described above. The liquid resulting from the hydrolysis reaction typically containing C5, C6, C12 and oligomeric sugars, so called hydrolyzate, can be separated from the cellulose and, if present, lignin solids, and the sugars can be converted by, for example, fermentation, to various chemical products such as alcohols including ethanol. In addition to sugars however, hydrolyzate can also contain other compounds such as one or more of aliphatic acids, esters (acetate), phenolics that are different compounds obtained from lignin hydrolysis, and products of sugar dehydration, including the furan aldehydes, furfural and 5-hydroxymethyl furfural (5-HMF). Many or most of these other compounds have a negative impact on microorganisms and can inhibit fermentation of sugars by microorganisms to an alcohol such as ethanol. Detoxification of the hydrolyzate prior to fermentation, as will be discussed in more detail below, can be used to avoid or minimize inhibition caused by toxic compounds present in the hydrolyzate.

Any suitable hydrolysis process can be used to prepare hydrolyzates, including acid hydrolysis and base hydrolysis. Acid hydrolysis is a relatively inexpensive and can be a fast method and can suitably be used. A concentrated acid hydrolysis is suitably operated at temperatures of about 20° C. to about 100° C., and an acid strength in the range of about 10% to about 93% by weight of the acid in the liquid phase, for example, about 10% to about 20, about 21% to about 30%, about 31% to about 40%, about 41% to about 55%, about 56% to about 70%, about 71% to about 85%, about 86% to about 93% by weight of the acid in the liquid phase.

Dilute acid hydrolysis is a simpler process, but is optimal at higher temperatures, for example at about 100° C. to about 230° C., and generally higher pressure compared to concentrated hydrolysis. Different kinds of acids, with concentrations in the range of 0.001% to 10% by weight of the acid in the liquid phase, are suitable, For example, about 0.001%, about 0.01%, about 0.05%, about 0.1%, about 0.15%, about 0.2%, about 0.25%, about 0.3%, about 0.35%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about or about 10%, by weight of the acid in the liquid phase. Suitable acids, for either the concentrated or dilute hydrolysis include, for example, nitric acid, sulfurous acid, nitrous acid, phosphoric acid, perchloric acid, hydroiodic acid, hydrobromic acid, hydrofluoric acid, formic acid, acetic acid, hydrochloric acid, citric acid, and sulfuric acid. Sulfuric acid is a particularly useful acid for the hydrolysis step using either dilute or concentrated acid hydrolysis. A mixture of one or more acids, such as the acids listed above, can also be used.

Depending on the acid concentration, and the temperature and pressure under which the acid hydrolysis step is carried out, corrosion resistant equipment and/or pressure tolerant equipment may be needed.

The hydrolysis can be carried out for a time period ranging from about 2 minutes to about 10 hours, for example, about 3 to about 5 minutes, about 6 to about 10 minutes, about 15 to about 20 minutes, about 21 to about 25 minutes, about 26 to about 30 minutes, or about 0.5 hours, about 0.75 hours, about 1 hour, about 1.5 hours, about 2 hours, about 3 hours, about 4 hours, about 5 hours, about 6 hours, about 7 hours, about 8 hours, about 9 hours, or about 10 hours, or any range bounded by any two of the foregoing values. For example, the time period for the hydrolysis can be 1 minute to 2 hours, 2 minutes to 15 minutes, 2 minutes to 2 hours, 15 minutes to 2 hours, 30 minutes to 2 hours, 10 minutes to 1.5 hours, or 1 hour to 5 hours.

The hydrolysis can also include, either with or without an acid treatment, and either before or after such acid treatment, a heat or pressure treatment or a combination of heat and pressure, for example, treatment with steam, for about 0.5 hours to about 10 hours, for example, about 0.5, about 1, about 1.5, about 2, about 3, about 3.5, about 4, about 5, about 6, about 7, about 8, about 9, or about 10 hours, or any range bounded by any two of the foregoing values. It can also include a two step hydrolysis process as described below.

The pressure for the hydrolysis reaction can be about 50 to about 250 psig, for example, about 50 to about 200 psig or about 70 to about 180 psig, or about 100 to about 160 psig.

Variations of acid hydrolysis processes are known in the art. For instance, the hydrolysis can be carried out by subjecting the feedstock to a two step process. The first is a chemical hydrolysis step suitably carried out in an aqueous medium at a temperature and a pressure chosen to effectuate primarily depolymerization of hemicellulose without achieving significant depolymerization of cellulose into glucose. This step yields a slurry in which the resulting liquid aqueous phase contains dissolved monosaccharides and soluble and insoluble oligomers of hemicellulose resulting from depolymerization of hemicellulose, and a solid phase containing cellulose and, if present in the feedstock, lignin. See, for example, U.S. Pat. No. 5,536,325. In one embodiment of a two step hydrolysis of a feedstock, sulfuric acid is utilized to effect the first hydrolysis step. After the sugars are separated from the first-stage hydrolysis process, the second hydrolysis step is run under more severe condition to hydrolyze the more resistant cellulose fractions.

Another process for feedstock hydrolysis comprises processing a lignocellulosic feedstock by one or more stages of dilute acid hydrolysis using about 0.4% to about 2% of an acid; followed by treating the unreacted solid lignocellulosic component of the acid hydrolyzed material with alkaline delignification. See, for example, U.S. Pat. No. 6,409,841. Another process for feedstock hydrolysis comprises prehydrolyzing feedstock such as a lignocellulosic feedstock, in a prehydrolysis reactor; adding an acidic liquid to the solid lignocellulosic feedstock to make a mixture; heating the mixture to reaction temperature; maintaining reaction temperature for a period of time sufficient to fractionate the lignocellulosic feedstock into a solubilized portion containing at least about 20% of the lignin from the lignocellulosic feedstock, and a solid fraction containing cellulose; separating the solubilized portion from the solid fraction, and removing the solubilized portion while at or near reaction temperature; and recovering the solubilized portion.

Feedstock hydrolysis can also comprise contacting a feedstock with stoichiometric amounts of sodium hydroxide and ammonium hydroxide at a very low concentration. See Teixeira et al., 1999, Appl. Biochem. and Biotech. 77-79:19-34. Hydrolysis can also comprise contacting a lignocellulosic feedstock with a chemical, for example, a base, such as sodium carbonate or potassium hydroxide, at a pH of about 9 to about 14 at moderate temperature and pressure. See PCT Publication WO 2004/081185.

Ammonia hydrolysis can also be used to hydrolyze feedstock. Such a hydrolysis method comprises subjecting a feedstock to low ammonia concentration under conditions of high solids. See, for example, U.S. Patent Publication No. 20070031918 and PCT publication WO 2006/110901.

In one suitable embodiment of the hydrolysis step, comminuted, pressed and washed feedstock is partially hydrolyzed thereby converting most or all of the C5 hemicellulose polymers to, primarily, C5 sugars, such as xylose, and oligomeric materials. Some of the C6 cellulose polymer is also converted to C6 sugars, such as glucose.

The weight ratio of water to solids in the hydrolysis reaction can be about 1:1 to about 10:1, for example about 2:1 to about 5:1, or about 2:1 to about 3:1.

The hydrolysis can be accomplished using a number of different hydrolysis apparatus, such as in a stirred reaction vessel or in a plug flow reactor. The reactor can have vanes or baffles to promote agitation and establish good contact between an acidic or basic aqueous phase and the polymeric sugars in the feedstock. The hydrolysis reaction can be a batch process or a continuous process. It can be single stage or multiple stages, such as 2 or 3 or 4 stages of hydrolysis.

In one embodiment, comminuted, for example, shredded, feedstock is treated with steam to add water and elevate the temperature of the feedstock, for example, to a temperature of about 150° C. to about 200° C., for example, about 160° C., or about 170° C., or about 180° C. or about 190° C., to about 200° C. The steam treated feedstock is conveyed to a plug-screw feeder where it forms a cake within the feeder. The plug-screw feeder compresses the cake of shredded feedstock into a plug at the end of the screw where it may be treated with acid for the hydrolysis. For example an aqueous acid mixture can be sprayed onto and injected into the plug of feedstock using one or more devices such as nozzles, jets, spray bars or spray rings, and the like. The feedstock so treated is moved down through, for example, a vertical hydrolyser apparatus at a desired rate where it is heated to undertake the hydrolysis reaction as described above. The desired residence time in the vertical hydrolyser unit can be controlled, for example, using a conveying screw on the bottom of the hydrolyser apparatus. Water, or as described below, other liquids containing water, can be added to the hydrolyzer apparatus to achieve a desired ratio of water to solids in the hydrolysis reaction. The product from the hydrolysis reaction in the hydrolyser apparatus can be removed from the hydrolyser apparatus through an orifice or nozzle, for example, at the bottom or lower portion of the hydrolyzer apparatus. At this stage, the hydrolysis reaction mixture is at an elevated pressure such as for example, a pressure of about 50 psia to about 250 psia, for example, about 100 psia, or about 150 psia, or about 170 psia or about 200 psia, to about 250 psia. When released, the mixture can undergo a rapid depressurization resulting in what can be referred to a steam explosion whereby the particle size of the solids portion of the feedstock material is reduced further. The rapid depressurization can, for example, occur within one or more devices such as a blow cyclone. The depressurization can be to a pressure of about 10 psia to about 30 psia, for example, about 15 psia or about 20 psia, to about 30 psia.

The mixture of solids and liquids produced by the hydrolysis reaction, either with or without the rapid depressurization step, can be treated to separate the liquid from the solids thereof using one or more separation apparatuses for separating solids from liquids, such as filters, presses, such as screw presses, centrifuges and the like. For example, one or more, such as 2, 3 or 4 such separation apparatuses in any combination, can be arranged in series where the solids from the first device are sent to the second separation apparatus in series, and so on, until the desired separation of the liquid from the solids portion is achieved. For example, at least about 50 percent of the water in the mixture of solids and liquids produced by the hydrolysis reaction is removed by the separation process, or at least about 60 percent of the water, or at least about 70 percent, or at least about 80 percent of the water is removed. The water that is removed can contain at least about 50 percent of the soluble sugars, such as one or more of sucrose, glucose, fructose, and xylose, at least about 60 percent, or at least about 70 or 80 percent; that was in the mixture of solids and liquids produced by the hydrolysis reaction.

Prior to undertaking the separation of the solids from the liquids in the mixture of solids and liquids produced by the hydrolysis reaction, the mixture can be combined with, for example, the first juice obtained from the feedstock. The amount of such first juice combined with the mixture of solids and liquids produced by the hydrolysis reaction can be an amount to assist with the separation of the liquid from the solids of the mixture of solids and liquids produced by the hydrolysis reaction and so that the liquid portion that is separated contains the desired amounts of water soluble sugars. Stated in another way, the amount of first juice that is combined with the mixture of solids and liquids produced by the hydrolysis reaction can be an amount so that when the separation of the solids form the liquid portion is undertaken, the soluble sugars are effectively separated from the mixture and present in the liquid portion that is separated. For example, the amount of the first juice that can be combined with the mixture of solids and liquids produced by the hydrolysis reaction can be an amount such that the ratio of the volume of first juice to the volume of the mixture of solids and liquids produced by the hydrolysis reaction is about 1:1, to about 10:1, for example, about 3:1 or about 5:1 to about 10:1. The first juice can be combined with the mixture of solids and liquids produced by the hydrolysis reaction in any suitable manner such as in a stirred vessel or as a wash liquid in a solid/liquid separation apparatus that can be used to separate the solids from the liquids.

As stated above, the liquid portion that is recovered from the separation of the mixture of solids and liquids produced by the hydrolysis reaction, either with or without the prior combination of the mixture with the first juice or other liquid, is referred to as the hydrolyzate.

At this stage in the described embodiment of the invention, there is the liquid portion, or hydrolyzate, as described above, comprising water, and typically also contains water soluble sugars such as one or more or sucrose, glucose, fructose, and xylose, and the solids portion that was separated from the hydrolyzate. The solids portion comprises cellulose that was not hydrolyzed in the hydrolysis reaction and, if present in the feedstock, may comprise lignin.

The concentration of the individual compounds in the starting hydrolyzate depends, in part, on the feedstock from which the hydrolyzate is obtained and the method used to hydrolyze the feedstock, as well as hydrolysis conditions. In certain embodiments, the starting hydrolyzate comprises (a) total fermentable sugars at a concentration of about 30 g/L to about 160 g/L, about 40 g/L to about 95 g/L, or about 50 g/L to about 70 g/L; (b) furfural at a concentration of about 0.5 g/L to about 10 g/L, about 2.5 g/L to about 4 g/L, or about 1.5 g/L to about 5 g/L; (c) 5-HMF at a concentration of about 0.1 g/L to about 5 g/L, about 0.5 g/L to about 2.5 g/L or about 1 g/L to about 2 g/L (d) acetic acid at a concentration of about 2 g/L to about 17 g/L or about 11 g/L to about 16 g/L; (e) lactic acid at a concentration of about 0 g/L to about 12 g/L or about 4 g/L to about 10 g/L; (f) additional aliphatic acids, for example, succinic acid, formic acid, butyric acid and levulinic acid, at concentrations of about 0 g/L to about 2.5 g/L; and/or (g) phenolics at a concentration of about 0 g/L to about 10 g/L, about 0.5 g/L to about 5 g/L or about 1 g/L to about 3 g/L. In these embodiments, the starting hydrolyzate will be referred to herein as “1×”.

In other embodiments, the starting hydrolyzate can be more concentrated than 1×. For example, the starting hydrolyzates can be about 1.5-fold, about 2-fold, about 3-fold, about 4-fold, about 5-fold, about 6-fold, about 7-fold, about 8-fold, about 9-fold or about 10-fold more concentrated than 1×. In these embodiments, the starting hydrolyzate will be referred to as 1.5×, 2×, 3×, 4×, 5×, 6×, 7×, 8×, 9× and 10×, respectively.

In other embodiments, the starting hydrolyzate can be less concentrated than 1×. For example, the starting hydrolyzate can be about 0.1-fold, about 0.2-fold, about 0.3-fold, about 0.4-fold, about 0.5-fold, about 0.6-fold, about 0.7-fold, about 0.8-fold or about 0.9-fold as concentrated as 1×. In these embodiments, the starting hydrolyzate will be referred to as 0.1×, 0.2×, 0.3×, 0.4×, 0.5×, 0.6×, 0.7×, 0.8×, and 0.9×, respectively.

The concentration of the hydrolyzate can be adjusted prior to a subsequent detoxification process. Without intending to be bound by any theory of operation, the toxicity of the hydrolyzate is correlated to the concentration of individual and to a combination of certain compounds in the hydrolyzate, such as 5-HMF and acetic acid.

### Detoxification of the Hydrolyzate

As mentioned above, the hydrolysis reaction can produce one or more chemical compounds that can inhibit the conversion of the soluble sugars in the hydrolyzate to an alcohol, such as ethanol, by a fermentation process. Consequently, it is desirable to reduce the amount of or suitably eliminate these detrimental compounds in the hydrolyzate prior to subjecting the hydrolyzate to a process, such as a fermentation process, to convert the sugars contained therein to an alcohol such as ethanol. The detrimental compounds can, for example, and as already mentioned above, be one or more of an aldehyde such as furfural or 5-hydroxymethyl furfural, one or more aliphatic acids, one or more esters and one or more phenolic compounds.

Various methods of detoxification can be used, such as alkaline overliming. Suitable detoxification processes are also described, for example, in U.S. Provisional Patent Application Ser. No. 61/597,936, filed Feb. 13, 2012, and U.S. Provisional Patent Application Ser. No. 61/597,973, filed Feb. 13, 2012, sections of which are set forth in this application.

During the overliming process, the pH of the hydrolyzate is temporarily raised, usually at an elevated temperature, from a pH of, for example, approximately 2 to a pH of, for example, between 9 and 10 through the addition of an appropriate amount of calcium hydroxide, commonly referred to as lime. After some time, typically about 30 minutes, the pH of the hydrolyzate solution is lowered through the addition of acid to a pH suitable for fermentation using microorganisms. In the detoxification process, furan aldehydes are degraded and acids, both mineral and organic, are neutralized.

Overliming has been known for a long time (see for example, Leonard and Hajny, 1945, Ind. Eng. Chem., 37 (4):390-395) and still is considered an efficient detoxification method. However, a significant drawback of that method is the considerable amount of loss of fermentable sugars that occurs during detoxification. See, for example, Larsson et al., 1999, Appl. Biochem. Biotechnol. 77-79:91-103. The loss of fermentable sugars ultimately results in lower overall yields of fermentable products such as ethanol. In addition, the formation of insoluble calcium sulfate, commonly referred to as gypsum, during detoxification with calcium hydroxide requires that the gypsum be subsequently treated in an suitable manner. See, for example, Martinez et al., 2001, Biotechnol. Prog. 17(2):287-293. Gypsum formation can also cause fouling and pipeline clogging, which can increase significantly maintenance costs.

As used herein, the term “detoxification” refers to a process in which one or more compounds that are detrimental to a fermenting microorganism, referred to herein as “toxins,” are either totally, substantially or partly removed from a starting hydrolyzate and/or are totally, substantially or partly inactivated, thereby forming a detoxified hydrolyzate. As used herein, the phrase “detoxified hydrolyzate” refers to a hydrolyzate containing lower toxin levels and/or deactivated toxins, relative to the level of toxins and toxins in the hydrolyzate prior to the treatment in a detoxification process. The hydrolyzate prior to detoxification is referred to herein as a “starting hydrolyzate.”

Accordingly, detoxification of the starting hydrolyzate reduces the toxicity of a starting hydrolyzate, such as a starting hydrolyzate derived from a lignocellulosic feedstock, towards a fermenting organism. In certain aspects, the detoxification methods involve mixing a starting hydrolyzate with a base, such as a magnesium base, for example, one or more of, magnesium hydroxide, magnesium carbonate or magnesium oxide, for a period of time and under conditions that result in the production a detoxified hydrolyzate. Provided are detoxified hydrolyzates in which the quantity of the toxins that are deleterious to fermenting microorganisms is substantially reduced and/or the toxins are deactivated relative to the starting hydrolyzate. At the same time, the amount of fermentable sugars lost in the detoxification process is suitably minimized.

The most suitable detoxification methods of the present disclosure provide detoxified hydrolyzates in which a substantial portion of the furan aldehydes have been removed relative to the starting hydrolyzate. At the same time, the detoxification results in minimal loss of fermentable sugars. Therefore, the detoxification reactions can be highly selective towards elimination of furan aldehydes. In certain embodiments, the methods disclosed herein result in the production of a detoxified hydrolyzate with at least 70%, at least 80%, at least 85%, at least 90%, at least 92%, at least 93%, at least 95% or at least 99% of the fermentable sugars present in the starting hydrolyzate and no greater than 70%, no greater than 60%, no greater than 50%, no greater than 40%, no greater than 30%, no greater than 20% or no greater than 10% of the furan aldehydes present in the staring hydrolyzate. In particular embodiments, detoxification methods of the present disclosure provide a detoxified hydrolyzate with (a) at least 90% of the total fermentable sugars present in the starting hydrolyzate and no greater than 50% of the furan aldehydes present in the starting hydrolyzate; (b) at least 90% of the total fermentable sugars present in the starting hydrolyzate and no greater than 40% of the furan aldehydes present in the starting hydrolyzate; (c) at least 90% of the total fermentable sugars present in the starting hydrolyzate and no greater than 30% of the furan aldehydes present in the starting hydrolyzate; (d) at least 90% of the total fermentable sugars present in the starting hydrolyzate and no greater than 20% of the furan aldehydes present in the starting hydrolyzate; (e) at least 80% of the total fermentable sugars present in the starting hydrolyzate and no greater than 50% of the furan aldehydes present in the starting hydrolyzate; (f) at least 80% of the total fermentable sugars present in the starting hydrolyzate and no greater than 40% of the furan aldehydes present in the starting hydrolyzate; (g) at least 80% of the total fermentable sugars present in the starting hydrolyzate and no greater than 30% of the furan aldehydes present in the starting hydrolyzate; or (h) at least 80% of the total fermentable sugars present in the starting hydrolyzate and no greater than 20% of the furan aldehydes present in the starting hydrolyzate.

As described above, the starting hydrolyzate can be concentrated prior to detoxification.

In various embodiments, the detoxification of the hydrolyzate can be carried out at a temperature of about 90° C. or less, for example, about 25° C. to about 90° C. The detoxification process can be carried out, for example, at about 30° C., about 35° C., about 40° C., about 45° C., about 50° C., about 55° C., about 60° C., about 65° C., about 70° C., about 75° C., about 80° C., about 85° C., or about 90° C. In specific embodiments, the detoxification process is carried out at a temperature in the range bounded by any two of the foregoing temperatures, for example, at a temperature of about 40° C. to about 60° C., about 40° C. to about 70° C., about 40° C. to about 55° C., about 40° C. to about 50° C., about 45° C. to about 50° C., about 45° C. to about 55° C., about 50° C. to about 55° C., about 35° C. to about 65° C., etc. Advantageously, the detoxification process is carried out at a temperature of about 40° C. to about 60° C., which allows the detoxification reactions to occur at a commercially feasible rate while minimizing the loss of fermentable sugars, and thereby increasing the yield of fermentation products such as ethanol.

The hydrolyzate detoxification process is typically carried out at a pH of about 6.2 to about 9.5, for example at a pH of about 6.5, about 7, about 7.5, about 8, about 8.5, about 9.0 or about 9.5. In specific embodiments, the pH is in the range bounded by any of the two foregoing values, such as, but not limited to, a pH of about 6.5 to about 8, about 6.5 to about 7.5, about 7 to about 8, or about 7 to about 7.5. It will be understood that the pH of the hydrolyzate solution depends on the concentration of the base and the temperature of the solution. In embodiments where hydrolyzate detoxification is carried out using magnesium hydroxide, the solubility of the magnesium hydroxide decreases with increasing temperature. Therefore, for a given amount of magnesium hydroxide added to the hydrolyzate solution, the equilibrium pH decreases as the temperature is increased, all other variables being constant. The pH of the solution can decrease slightly as the detoxification process progresses owing to the consumption of base in reaction with sugars and furans. Additional base can be added to the hydrolyzate to adjust the pH during the course of the detoxification reaction.

In certain embodiments, there is a method of reducing the toxicity of a lignocellulosic hydrolyzate towards a fermenting organism, comprising the step of mixing a starting lignocellulosic hydrolyzate solution, said starting lignocellulosic hydrolyzate solution comprising a mixture of fermentable sugars, furan aldehydes and aliphatic acids, with a magnesium base for a period of time of at least 1 hour, at least 4 hours, at least 10 hours or at least 20 hours at a temperature between 40° C. and 70° C. and at a pH of between 6.5 and 8. In particular embodiments, the magnesium base is magnesium hydroxide.

The detoxification methods can comprise mixing a starting lignocellulosic hydrolyzate solution with a magnesium base for a period of time and under conditions that result in the production of a detoxified hydrolyzate solution. The amount of time suitable to perform the detoxification process depends on a number of factors, including the chemical composition of the hydrolyzate, the concentration of the hydrolyzate solution, the reaction temperature, the pH of the hydrolyzate solution, the total amount of magnesium base added, the stirring rate, and the type of reactor being used. The hydrolyzate detoxification process is typically carried out for a period of time of about 15 minutes to 80 about hours, and more typically between about 1 hour and about 40 hours. In specific embodiments, the detoxification process is carried out for a period of about 1 hour to about 30 hours, about 1.5 hours to about 20 hours, about 2 hours to about 12 hours, about 3 hours to about 9 hours, about 4 hours to about 10 hours, or about 6 hours to about 9 hours. This process is applicable for batch and continuous vessel treatments.

The total amount of a magnesium base added to hydrolyzate solution 1× can be about 2 grams per 1 kilogram hydrolyzate (2 g/l kg hydrolyzate) to about 200 grams per 1 kilogram hydrolyzate (200 g/l kg hydrolyzate). For instance, the total amount of magnesium base added to the hydrolyzate solution can be about 40 g/l kg hydrolyzate, about 80 g/l kg hydrolyzate, about 100 g/l kg hydrolyzate, about 120 g/l kg hydrolyzate, about 140 g/l kg hydrolyzate, or about 160 g/l kg hydrolyzate. The magnesium base can be added to the hydrolyzate solution in a single step, in multiple portions or continuously throughout the course of the detoxification process. In specific embodiments, the total amount of magnesium base added to the hydrolyzate solution is in the range bounded by any of the two foregoing embodiments, such as, but not limited to, about 40 g/l kg hydrolyzate to about 160 g/l kg hydrolyzate, about 40 g/l kg hydrolyzate to about 120 g/l kg hydrolyzate, about 80 g/l kg hydrolyzate to about 160 g/l kg hydrolyzate, about 80 g/l kg hydrolyzate to about 140 g/l kg hydrolyzate, or about 140 g/l kg hydrolyzate to about 160 g/l kg hydrolyzate. For more concentrated hydrolyzate solutions, for example, 4×, the amount of magnesium base sufficient to raise the pH to the desired level would be increased relative to hydrolyzate solution 1×. For less concentrated hydrolyzate, for example, 0.5×, the amount of magnesium base sufficient to raise the pH to the desired level would be decreased relative to hydrolyzate solution 1×.

Provided further are methods for continuously reducing the quantity of toxins in a hydrolyzate, comprising the steps of flowing a first continuous stream of a hydrolyzate into a continuous reactor or a series of continuous reactors, flowing a second continuous stream of a solution of a base, such as a magnesium base, into the continuous reactor or the series of continuous reactors, mixing the hydrolyzate with the base in the continuous reactor for a period of time sufficient to reduce the quantity of toxins in the hydrolyzate, and flowing the hydrolyzate out of the continuous reactor.

One suitable process comprises temporarily increasing the pH of the hydrolyzate, suitably while at an elevated temperature, to a pH of about between 9 and 10 by combining calcium hydroxide with the hydrolyzate. After a suitable amount of time, for example, about 30 minutes, at this pH and, optionally, at an elevated temperature, the pH of the hydrolyzate is lowered by, for example, the addition of a suitable acid such as sulfuric acid, to a pH that is acceptable for fermentation of the sugars in the detoxified hydrolyzate to fermentation products such as ethanol. Another suitable hydrolyzatee detoxification process comprises increasing the pH of the hydrolyzate to about 5 to about 6 using, for example, one or more basic compounds, such as one or more of ammonium hydroxide, sodium hydroxide, potassium hydroxide, calcium hydroxide, magnesium hydroxide, magnesium carbonate or magnesium oxide. The aforementioned magnesium-containing bases are advantageous. The amount of base that is used is an amount that achieves the desired pH. The temperature for the first step can be about 25° C. or greater and can be up to, for example, about 90° C. The detoxification process can be carried out for about 15 minutes to about 40 hours.

It is also possible to use a detoxification procedure that comprises two steps using a first base for the first step and a second base for the second step. The first step of the detoxification process can comprise combining the hydrolyzate with a first base or first mixture of bases to increase the pH of the hydrolyzate to a pH of, for example, about 3 to about 9, for example, to a pH of about 3 to about 4, about 3 to about 5, or about 4 to about 6. In the second step the hydrolyzate after treatment in the first step is combined with a second base or second mixture of bases at a pH of about 7 to about 10, for example at a pH of about 7, about 8, about 9 or about 10. In specific embodiments, the pH is in the range bounded by any of the two foregoing embodiments, for example, a pH of about 7 to about 9, about 8 to about 9, about 8 to about 10. The first base can be any suitable base and can be, for example, one or more of magnesium hydroxide, magnesium carbonate or magnesium oxide. The second base can be the same as the first base but can also be, for example, one or more of ammonium hydroxide, ammonia, sodium hydroxide, potassium hydroxide, calcium hydroxide. The amount of base that is used is an amount that achieves the desired pH. The temperature for the first step can be about 25° C. or greater and can be up to, for example, about 90° C., for example about 35° C. to about 55° C. The temperature for the second step can be about 4° C. or greater and can be up to, for example, about 90° C., for example about 40° C. to about 80° C., about 40° C. to about 70° C., about 40° C. to about 60° C., about 40° C. to about 50° C., about 50° C. to about 55° C., about 45° C. to about 50° C., or about 47° C. to about 50° C. The first step of the two step detoxification process can be carried out for about 1 to about 60 minutes, for example, about 20 to about 60 minutes, and the second step for about 30 minutes to about 20 hours, for example, for about 1 hour to about 3 hours.

The detoxification methods can be performed in any suitable vessel, such as a batch reactor or a continuous reactor, for example, a continuous stirred tank reactor (CSTR) or a plug flow reactor (PFR). A continuous reactor allows for continuous addition and removal of input materials, for example, hydrolyzate and magnesium base slurry, as the detoxification reaction progresses. The suitable vessel can be equipped with a means, such as impellers, for agitating the hydrolyzate solution. Reactor design is discussed in, for example, Lin, K.-H., and Van Ness, H. C. (in Perry, R. H. and Chilton, C. H. (eds), Chemical Engineer's Handbook, 5th Edition (1973) Chapter 4, McGraw-Hill, NY.

The detoxification processes can be carried out in a batch mode. The methods typically involve combining the hydrolyzate solution and the magnesium base (or magnesium base slurry) in the reactor. The hydrolyzate solution and the magnesium base can be fed to the reactor together or separately. Any type of reactor can be used for batch mode detoxification, which simply involves adding material, carrying out the detoxification process at specified conditions (for example temperature, dosage and time) and removing the detoxified hydrolyzate from the reactor.

Alternatively, the detoxification processes can be carried out in a continuous mode. The continuous processes of the disclosure advantageously reduces the need to stop and clean reactors and accordingly can be carried out in continuous mode, e.g., for periods of several days or longer (e.g., a week or more) to support an overall continuous process. The methods typically entail continuously feeding a hydrolyzate solution and base slurry to a reactor. The hydrolyzate and the base slurry can be fed together or separately. The resultant mixture has a particular retention or residence time in the reactor. The residence time is determined by the time to achieve the desired level of detoxification following the addition of the hydrolyzate and the base to the reactor. Following the detoxification process, the detoxified hydrolyzate exits the reactor and additional components (e.g., hydrolyzate and base slurry) are added to the reactor. Multiple such reactors can be connected in series to support further pH adjustment during an extended retention time and/or to adjust temperature during an extended retention time.

For detoxification in continuous mode, any reactor can be used that allows equal input and output rates, e.g., a continuous stirred tank reactor or plug flow reactor, so that a steady state is achieved in the reactor and the fill level of the reactor remains constant.

The detoxification processes disclosed herein can be carried out in semicontinuous mode. Semicontinuous reactors, which have unequal input and output streams that eventually require the system to be reset to the starting condition, can be used.

The present disclosure provides methods of continuously detoxifying a feedstock obtained from a lignocellulosic feedstock. The steps of the continuous detoxification process include flowing a first continuous stream of a hydrolyzate into a continuous reactor, flowing a second continuous stream of a solution of a magnesium base into the continuous reactor, mixing the hydrolyzate with the magnesium base in the continuous reactor for a period of time sufficient to reduce the quantity of toxins in the hydrolyzate, and flowing the hydrolyzate out of the reactor.

Adequate mixing of the hydrolyzate solution following addition of the base can improve the rate of dissolution of the base and ensure that the pH remains substantially homogeneous throughout the solution. For instance, ideal mixing will avoid the formation of local pockets of higher pH, which can result in lower selectivity for furan elimination. Mixing speeds of between 100 revolutions per minute (rpm) and 1500 rpm can be used to ensure sufficient mixing of the hydrolyzate solution. For instance, mixing speeds of 100 rpm, 200 rpm, 400 rpm, 800 rpm and 1500 rpm can be used. In specific embodiments, mixing is carried out at speeds bounded by any two of the foregoing mixing speeds, such as, but not limited to from 100 rpm to 200 rpm, from 100 rpm to 400 rpm, from 200 rpm to 400 rpm, from 400 rpm to 800 rpm or from 800 rpm to 1,500 rpm. In other embodiments, intermittent mixing regimes can be used where the rate of mixing is varied as the detoxification process progresses. Mixing of the hydrolyzate solution can be accomplished using any mixer known in the art, such as a high-shear mixer, paddle mixer, magnetic stirrer or shaker, vortex, agitation with beads, and overhead stirring.

### Propagation of a Fermentation Microorganism

Although a mixture of fermentation microorganisms, such as one or more different kinds of yeasts, can be used, suitably, a single fermentation organism such as a single kind of yeast that is capable of fermenting both C5 and C6 sugars is used in embodiments of this invention. The microorganism can be a wild type of microorganisms or a recombinant microorganisms, and can include, for example, Escherichia, Zymomonas, Saccharomyces, Candida, Pichia, Streptomyces, Bacillus, Schizosaccharomyces, Dekkera, Brelanomnyces, Kluvveromyces, Issatchenkia, Hansenula, Pachysolen, Torulaspora, Zygosaccharomyces, Yarrowia, Lactobacillus, and Clostridium. Particularly suitable species of fermenting microorganisms include Escherichia coli, Zymomonas mobilis, Bacillus stearothermophilus, Saccharomyces cerevisiae, Clostridia thermocellum, Thermoanaerobacteriumn saccharolyticum, and Pichia stipitis. Genetically modified strains of E. coli or Zymomonas mobilis can be used for ethanol production (see, for example, Underwood et al., 2002, Appl. Environ. Microbiol. 68:6263-6272 and US 2003/0162271 A1).

Suitable fermentation organisms include, for example, S. cerevisiae. S. carlsbergensis, S. pastorianus, BioTork strain SC48-EVG51, Schizosaccharomyces pombe, D. bruxellensis, D. Anomala, B. bruxellensis. B. anomalus, B. custerianus, B. naardensis, B. nanus, K. marxianus, K. lactis, C. sonorensis, C. methanosorbosa, C. ethanolica, C. maltose, C. ropicalis, C. albicans, C. stellate, C. shehatae, I. orientalis (also known as Pichia kudriavzevii and the anamorph form (asexual form) known as Candida krusei), ATCC 3196, ATCC PTA-6658, Issalchenkia Kudryavtsev, Cargill strain 1822, Cargill strain 3556, Cargill strain 3085, Cargill strain 3849, Cargill strain 3859, H. polymorpha ML3, H. polymorpha ML9, H. polymorpha ML6, H. polymorpha ML8, H. polymorpha N95, P. tannophilus, P. tannophihus strain NRRL 2460, P. tannophilus strain I fGB 0101, P. stipitis (now known as Schefersomyces stipitis), Scheffersomyces stipilis strain CBS 6054, Scheffersomyces stipilis NRRL 7124, Scheffersomyces stipitis NRRL 11545, P. fermentans, P. faleiformis, P. sp. YB-4149, P. deserticola, P. membranifaciens, P. galeiformis, P. segobiensis. P. segobiensis strain NRRL 11571, T. delbruekii, Z. bailii, and Y. lipolytica The microorganism can be propagated in one or more separate vessels located at or near the vessels used to undertake the fermentation steps in the embodiments of this invention. The microorganism selected for the fermentation can be propagated in one or more suitable, hygienic vessels, at a pH of about 4 to about 7, or about 4 to about 5, and at a temperature of about 30° C. to about 45° C., or about 30° C. to about 34° C. Aeration and agitation can be used to assist with the propagation. The specific growth rate of the microorganism can be about 0.05 hr⁻¹ to about 1.7 hr⁻¹, or about 0.14 to about 1.20 hr⁻¹. For example, about 0.1, or about 0.2, or about 0.3 hr⁻¹ For example, the microorganism can be propagated in a series of successively larger vessels by extracting a portion of the contents of a vessel and using it to inoculate the contents of a larger vessel. In that way, a large supply of the microorganism can be prepared. For example, inoculate from vessels ranging from about 10 to about 30 gallons can be used to inoculate the contents of vessels of about 400 to about 600 gallons, and inoculate from these vessels can be used to inoculate the contents of vessels of about 10,000 to about 15,000 gallons, and inoculate from these vessels can be used to inoculate the contents of vessels of about 20,000 to about 30,000 or 40,000 gallons. The microorganism, for example, the selected yeast, concentration in the vessels can be, for example, about 1×10⁷ CFU/mL to about 10×10⁷ CFU/mL. A base such as ammonium hydroxide can be used to maintain the desired pH of the mixture containing the microorganism and a sugar, such as glucose, can be used as the energy source for the microorganism propagation. Additionally, in order to promote the adaptation of the fermentation microorganism to the hydrolyzate, a portion of the hydrolyzate can be added to the vessels or vessels used to propagate the microorganism.

### Fermentation of the Hydrolyzate

The fermentation of the mostly C5 sugars in the hydrolyzate, especially the detoxified hydrozylate, to fermentation products can be carried out by one or more appropriate fermenting microorganisms in single or multistep fermentations to produce a first fermentation mixture.

The fermentation of the sugars in the hydrolyzate can be carried out in a minimal media with or without additional nutrients such as vitamins and corn steep liquor (CSL). The fermentation can be carried out in any suitable fermentation vessel. For instance, fermentation can be carried out in one or more, for example about 2 to about 10 large vessels, each having a capacity of about 50,000 to about 1,000,000 gallons. The fermentation process can be performed as a batch, fed-batch or as a continuous process. The amount of inoculate that is added to the fermentation mixture can be about 1 to about 2 grams dry cell weight (DCW) per liter of fermentation broth. of the microorganism. The starting pH of the fermentation mixture can be about 3.5 to about 8, and more typically from about 4 to about 7. The pH is suitably maintained at about 4 to about 6.5. A suitable base, such as for example, ammonium hydroxide, can be used to maintain a desired pH. The fermentation is generally carried out at a temperature of about 20° C. to about 40° C., and more typically about 25° C. to about 35° C. In particular embodiments, the fermentation is carried out for a period of time of about 5 to about 90 hours, about 10 to about 70 hours, or about 25 to about 50 hours. During this fermentation, soluble sugars in the hydrolyzate are converted, that is, fermented, to an alcohol such as ethanol to form a first fermentation mixture. At least part of the soluble sugars are fermented. For example, about 99 to about 5 weight percent of the sugars in the hydrolyzate are fermented to an alcohol such as ethanol. For example, about 95 to about 99, or about 90 to about 94, about 85 to about 89, about 80 to about 84, about 75 to about 79, about 70 to about 74, about 65 to about 69, about 60 to about 64, about 55 to about 59, about 50 to about 54, about 45 to about 49, about 40 to about 44, about 35 to about 39, about 30 to about 34, about 25 to about 29, about 20 to about 24, about 15 to about 19, about 10 to about 14, about 5 to about 9 weight percent of the soluble sugars in the hydrolyzate are fermented to an alcohol such as, for example, ethanol. The first fermentation mixture can have a concentration of about 0.5 to about 4, or about 0.5 to about 3, or about 2 to about 4, weight by volume % ethanol.

In embodiments of this invention, the fermentation of the hydrolyzate, and particularly the detoxified hydrolyzate, is advantageously conducted so that only part of the sugars in the hydrozylate are fermented, for example, the amount of sugars specified above. As will be discussed in more detail below, the first fermentation mixture can be combined with the solids portion recovered after the hydrolysis step, that is the solids portion comprising cellulose. That solids portion is subjected to saccharification to produce one or more sugars and those sugars are fermented in a second fermentation step to produce a second fermentation mixture. Any unfermented sugars remaining in the first fermentation mixture and that are combined with the solids portion, can be fermented along with the sugars produced by the saccharification. Consequently, the first fermentation need not be carried out for a time or under conditions that complete the fermentation of all the sugar in the hydrolyzate. By not conducting the fermentation of the hydrolyzate to completion then, for example, a smaller fermentation vessel can be used for the fermentation of the hydrolyzate, and/or less microorganism, such as a yeast, can be used, and/or the fermentation can proceed for less time, compared to the process where the fermentation is conducted to ferment all of the sugars in the hydrolyzate to an alcohol such as ethanol. For example, in a different process whereby at least part of the hydrolyzate is not combined with the solids portion.

### Saccharification Proteins

As used in this application, the term “saccharification or “saccharify” means the conversion, for example, conversion by enzymes, of cellulose into one or more sugars, such as glucose. Although not intending to be bound by any theory of operation, the solid, cellulosic portion of the product from the above-described feedstock hydrolysis and separation step can be treated to undergo a saccharification step whereby the cellulose polymer is converted to sugars. These sugars can be fermented to produce an alcohol such as ethanol. In some embodiments the fermentation can take place in the same vessel as the saccharification and thus this element of the process can be a simultaneous saccharification and fermentation of the cellulosic material in the solids portion. This simultaneous saccharification and fermentation can be effected by an enzyme or enzymes used in combination with a microorganism for fermenting a sugar to ethanol. The enzymes or enzymes convert the polymeric cellulosic material to sugar molecules, such as glucose, which then can be fermented to an alcohol such as ethanol. By using microorganisms, such as yeast, to perform the fermentation of the sugars to ethanol, the enzymes that effect the saccharification are not inhibited or are inhibited less so by the increase in concentration of the sugars in the mixture where the simultaneous saccharification and fermentation is occurring. Again, without intending to be bound by any theory of operation, suitably, the enzymes that are used to undertake the saccharification of the cellulosic material can be a combination of cellulases such as a cellbiohydrolase (CBH), an endoglucanase (EG) and a beta-glucosidase (β-G). The CBH activity is along crystalline portions of the cellulose fiber and produces cellobiose, the dimer of glucose, as the main product of its action on the cellulose fiber. The CBH activity is inhibited by the accumulation of cellobiose. The β-G enzyme cleaves the cellobiose to glucose thereby reducing the concentration of cellobiose as an inhibitor to the activity of CBH. The EG enzyme hydrolyzes the cellulose fiber in amorphous regions of the fiber thereby producing new free ends of crystalline cellulose portions of the cellulose fiber and thereby providing new substrate for the CBH. The increase in the concentration of glucose inhibits the activity of β-G. However, by introducing a microorganism to the mixture, such as yeast, that can convert the glucose to ethanol, the concentration of sucrose molecules can be reduced thereby relieving the inhibitor) effect of the glucose on the β-G. For this reason, a simultaneous saccharification and fermentation can be beneficial compared to the embodiment where the saccharification is conducted first and then the resulting sugars, either in the same vessel used for the saccharification or in a different vessel, can be fermented to an alcohol such as ethanol.

Enzymes suitable for saccharification of the solids portion comprising cellulose that is recovered from the feedstock hydrolysis step includes cellulases, hemicellulases, including, for example, xylanases, mannanases, and beta-xylosidases. Other proteins can be used with the cellulases or hemicellulases that enhance saccharification by cellulase or hemicellulases, such as carbohydrate esterases, for example, acetyl xylan esterases and ferulic acid esterases, and laccases, which are believed to act on lignin, and non-enzymatic proteins such as swollenins which are thought to swell the cellulose to make it more accessible to cellulases.

A cellulase cocktail suitable for saccharification of the cellulosic material in the solids portion recovered from the hydrolysis step can include one or more cellobiohydrolases, endoglucanases and/or β-glucosidases. Cellulase cocktails can be compositions comprising two or more cellulases. Cellulase cocktails can contain the microorganism culture that produced the enzyme components. A cellulase cocktail can be a crude fermentation product of the microorganisms such as the fermentation broth that has, for example, been separated from the microorganism cells and/or cellular debris by, for example, centrifugation and/or filtration. The enzymes in such a broth can be optionally diluted, concentrated, partially purified, purified and/or dried.

Cellulase cocktails that are suitable for the processes of this invention can include one or more proteins not normally produced by a cellulase-producing microorganism. The non-native proteins can be foreign or engineered proteins recombinantly co-expressed with other cellulase cocktail components by a cellulase-producing microorganism, for example, bacterium or fungus, or natively or recombinantly produced separately from other cellulase components, for example, in a bacterium, plant or fungus, and added to a cellulase cocktail. Mixtures of enzymes from different organisms can also be used in a cellulose cocktail.

Suitable cellulases include those of bacterial or fungal origin. Suitable cellulases include cellulases from the genera Bacillus, Pseudomonas, Trichoderma, Aspergillus, Ruminococcus, Clostridium, Chrysosporiuim, Hlumicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259. The Trichoderma reesei cellulases are disclosed in U.S. Pat. No. 4,689,297, U.S. Pat. No. 5,814,501, U.S. Pat. No. 5,324,649, WO 92/06221 and WO 92/06165. Bacillus cellulases are disclosed in U.S. Pat. No. 6,562,612. The Technische Universität München, Department of Microbiology, publishes on its website a list of cellulolytic bacterial species. As of the filing date of this application, there are 58 bacteria listed. Also, cellobiohydrolase I, cellobiohydrolase II, beta-glucosidase, and endoglucanase are suitable

Commercially available cellulases or cellulase cocktails that can suitably be used in the processes of this invention include, for example, CELLIC CTec (Novozymes), ACCELLERASE (Genencor), SPEZYME CP (Genencor), 22 CG (Novozymes), Biocellulase W (Kerry) and Pyrolase (Verenium), Novozyme-188 β-glucosidase (Novozymes), AlternaFuel® CMAX™ (Dyadic), AlternaFuel® 100P (Dyadic), AlternaFuel® 200P (Dyadic), AlternaFuel® CMAX3™ (Dyadic), Cellic CTec3 (Novozymes), Cellic CTec2 (Novozymes), Cellic CTec (Novozynmes), Cellic HTec3 (Novozymes), Accellerase® TRIO (Genencor).

Enzyme or enzyme cocktails can be used in doses ranging from about 5 μg to about 20 mg protein in the enzyme or enzyme cocktail per gram dry weight of the solids portion comprising cellulose that is recovered from the hydrolysis reaction. For example, about 5 μg, about 10 μg, about 20 μg, about 50 μg, about 100 μg, about 250 μg, about 500 μg, about 1 mg, about 2 mg, about 5 mg, about 10 mg, or about 20 mg of protein per grain dry weight of the solids portion comprising cellulose that is recovered from the hydrolysis reaction. In various embodiments, the dosage per gram dry weight of such solids portion is in a range bounded by any two of the foregoing embodiments, such as about 10 μg to about 250 μg, about 20 μg to about 500 μg, about 50 μg to about 250 μg, about 10 μg to about 100 μg, or about 20 μg to about 250 μg, about 100 μg to about 10 mg, about 250 μg to about 20 mg of protein per gramn dry weight of the solids portion comprising cellulose that is recovered from the hydrolysis reaction.

The term CTU as used herein refers to units of cellulase activity as measured using CELLAZYME T tablets (Megazyme, Co. Wickow, Ireland). The substrate in this assay is azurine-crosslinked Tamarind Xyloglucan (AZCL-Xyloglucan). This substrate is prepared by dyeing and cross-linking highly purified xyloglucan to produce a material which hydrates in water but is water insoluble. Hydrolysis by cellulase, for example, endo-(1-4)-b-D-glucanase, produces water soluble dyed fragments and the rate of release of these (increase in absorbance at 590 nm) can be related directly to enzyme activity. One CTU is defined as the amount of enzyme required to release one micromole of glucose reducing sugar-equivalents per minute from barley β-glucan (10 mg/mL) at pH 4.5 and 40° C. A mass of 1 mg of total protein of a T. reesei cellulase cocktail (as measured by the Bradford assay) corresponds to approximately 27.4 CTU.

Cellulases are preferably used in at doses of about 10 CTU to about 500 CTU cellulase per gram dry weight of the solids portion comprising cellulose that is recovered from the hydrolysis reaction. For example, about 10 CTU, about 20 CTU, about 30 CTU, about 40 CTU, about 50 CTU, about 60 CTU, about 80 CTU, about 100 CTU, about 125 CTU, about 150 CTU, about 175 CTU, about 200 CTU, about 250 CTU, about 300 CTU, about 400 CTU or about 500 CTU. In various embodiments, the amount of cellulase per gram dry weight of the solids portion comprising cellulose that is recovered from the hydrolysis reaction is in a range bounded by any two of the foregoing embodiments, such as, for example, about 10 CTU to about 200 CTU, about 20 CTU to about 400 CTU, about 40 CTU to about 250 CTU, about 10 CTU to about 100 CTU, or about 20 CTU to about 250 CTU.

### Saccharification of Cellulosic Solids and Fermentation of C6 Sugars

The solid portion recovered from the feedstock hydrolysis and separation step can be mixed with one or more liquid streams, such as, for example, at least part of the first fermentation mixture, to form slurry of the solids portion in the selected liquid. Before entering the vessel or vessels for the saccharification, the solids portion comprising cellulose can be mixed with such one or more liquid steams in a slurry tank. For example, the weight ratio of the liquid, such as the first fermentation mixture, to the solids portion can be about 5:1 to about 20:1, for example, about 5:1 to about 10:1. The saccharification and fermentation, either separately or together, can be run in one or more, for example, about 2 to about 10, or about 4 to about 8, suitable vessels each having a capacity of, for example, about 100,000 to about 1,000,000 gallons, or about 200,000 to about 600,000 gallons. These vessels, for saccharification of solids portion comprising cellulose and for the fermentation of the sugars produced by the saccharification, can be made of any suitable material and can be, for example, stainless steel. The vessels can be stirred or otherwise agitated by a suitable stirrer or device to agitate the contents to provide adequate mixing. The fermentation of the sugars produced by the saccharification can be conducted anaerobically. If the first fermentation mixture is used as the liquid for preparing the slurry of the solids portion, then the slurry can contain a fermentation organism for fermenting C6 sugars produced by the saccharification process. The saccharification of the solids portion can be accomplished enzymatically by permitting the enzyme or enzymes to interact with the cellulosic solid portion recovered from the feedstock hydrolysis and separation step.

The saccharification and fermentation processes, for example, when conducted as a SSF, can be performed as a batch, fed-batch or as continuous processes. It can be conducted in stages of addition of the solids portion to the reactor.

The amount of microorganism, such as a yeast, added to the saccharification and fermentation mixture can be about 0.5 to about 50 grams DCW per liter of fermentation broth. However, depending on the specific fermentation process and the selected feedstock, the amount of microorganism added can be different. For example, a greater amount of microorganism can be added.

The pH of the saccharification and fermentation mixture can be about 3.5 to about 7.0, and more typically from about 4.5 to about 6.0. The pH can be suitably maintained at about 4.5 to about 5.5. The saccharification and fermentation can be carried out at a temperature of about 30° C. to about 45° C., or to about 60° C., or to about 65° C., or higher, and more typically about 32° C. to about 39° C. In particular embodiments, the saccharification and fermentation can be carried out for a period of time of up to about 72 hours, up to about 42 hours, or up to about 24 hours. During this saccharification and fermentation, cellulose is saccharified to sugars such as glucose and sugars such as glucose are fermented to an alcohol such as ethanol to form a second fermentation mixture.

A base, such as one or more of magnesium hydroxide, ammonia, alkali metal hydroxides or metal carbonates or metal oxidescan be added to one or more of the slurry tank, if used, and the saccharification and fermentation vessel or vessels, to neutralize residual acid that may be included with the solids portion comprising cellulose that is recovered from the hydrolysis reaction. Optionally, cellulase enzymes, such as, Trichoderma reesei derived enzymes, can be added to the slurry tank in order to reduce the viscosity of the slurry. If the same enzyme or enzyme cocktail is used in the slurry tank as for the saccharification, then at least some and up to about 20 percent, for example up to about 18, or about 16, or about 14, or about 12 or about 10 percent of the total enzyme or enzyme cocktail used for the saccharification can be added to the slurry tank.

Optionally, the portion of the first fermentation mixture that can be combined with solids portion comprising cellulose that is recovered from the hydrolysis reaction can be heat treated prior to being combined with such solids portion to ensure any contaminating microorganisms in the first fermentation mixture are inactivated or killed. In one embodiment, heat treatment is performed using an in-line heat exchanger elevating the temperature of the first fermentation mixture as it passes through to a temperature that can be in the range of about 70° C. to about 100° C., for example about 80° C. to about 85° C., for a period of time, for example, of about 1 second to about 60 seconds, or for about 45 seconds. This heat treatment step can deactivate or kill undesirable competing microorganisms, if present, in the first fermentation mixture, and they will not, therefore, be added to the second fermentation step where the sugars produced by the saccharification are fermented to an alcohol, such as ethanol.

Optionally, beta hops acids can be added to the mixture for the fermentation of the sugars produced by the saccharification. Beta hops acids have a strong bacteriostatic effect against Gram positive bacteria and favor yeast such as S. cerevisiae. Again, competing microorganisms that might be present in the primary fermentation mixture will be rendered inactive without having to subject the first fermentation mixture to a heat treatment step prior to secondary fermentation.

The simultaneous saccharification and fermentation can occur at, for example, a pH 5.0, at 35° C. and with a residence time of for example 30 hours. T. reesei enzyme preparation can be added up to 225 CTU/g solids. The slurry of the first fermentation mixture and solids portion comprising cellulose can be fed at a solids concentration ranging from 14% to 20% depending on the use of 7: reesei preparation in the slurry tank for viscosity reduction. The secondary (C6) fermentation can produce a second fermentation mixture having 4% w/v to 12% w/v ethanol, for example, 4% w/v to 9% w/v ethanol, or 4% w/v to 6% w/v ethanol.

In one embodiment, a volume of the primary fermentation mixture can be transferred to the vessel or vessels for the simultaneous saccharification and fermentation. An amount of a cellulolytic enzyme cocktail is added to the vessel or vessels and a slurry of the solid portion of the product from the hydrolysis reaction and first fermentation mixture is fed to the vessel or vessels over a period of 5 to 20 hours. During this time the pH of the slurry is adjusted by the addition of base to raise the pH above its average of 1.5 to 2.5 such that the addition of the low pH slurry to the vessel or vessels used for the saccharification and fermentation does not lower the pH of the mixture in the vessel or vessels below a desired pH of 5.0 to 5.5. Also the temperature of the slurry is controlled such that the addition of the slurry does not take the mixture in the vessel or vessels out of the range of 32 to 38° C. The enzyme cocktail is fed to the process at a rate of 2 to 3% final working volume. Over the course of the fill and subsequent fermentation time, yeast will ferment soluble sugars such as one or more of glucose, fructose, xylose, and sucrose to ethanol and carbon dioxide. Simultaneously, the enzymes can act on the cellulose in the solids and through the action of the enzymes will liberate glucose. This glucose will also be fermented to ethanol and carbon dioxide. This process will continue to either complete uptake and utilization of the fermentable sugars or the timing of the process dictates that the simultaneous saccharification and fermentation and must be moved forward to free the vessels for a following batch.

Optionally, to drive the reaction forward in the saccharification and fermentation step, the pH can be variable. An optimal pH range for the saccharification enzymes can be about 5.0 to about 5.5. This is lower than the optimal pH for the fermentation organism which is about 6 to about 7. By alternating the pH from a lower range to a higher range and back again the fermentation step can be optimized. At the optimal pH conditions for saccharification enzymes more glucose can be generated. At the optimal pH conditions for the fermentation organism the liberated glucose can be more readily converted to ethanol. The overall ethanol yield in the fermentation step of the saccharification and fermentation can be enhanced, and the time for the saccharification and fermentation step can potentially be decreased.

### Recovery of Fermentation Products

After fermentation, the fermentation product, for example, ethanol can be separated from second fermentation mixture by any of the many conventional techniques known to separate ethanol from aqueous solutions. These methods include evaporation, distillation, azeotropic distillation, solvent extraction, liquid-liquid extraction, membrane separation, membrane evaporation, adsorption, gas stripping, pervaporation, and the like.

Fermentation products can be recovered using various methods known in the art. Products can be separated from other fermentation components by centrifugation, filtration, microfiltration, and nanofiltration. Products can be extracted by ion exchange, solvent extraction, or electrodialysis. Flocculating agents can be used to aid in product separation. As a specific example, bioproduced ethanol can be isolated from the fermentation medium using methods known in the art for ABE fermentations (see for example, Durre, 1998, Appl. Microbiol. Biotechnol. 49:639-648; Groot et al., 1992, Process. Biochem. 27:61-75; and references therein). For example, solids can be removed from the fermentation medium by centrifugation, filtration, decantation, or the like.

In one embodiment, a fermentation mixture is suitably transferred to a large holding vessel, where it can be stored before being forwarded to a distillation unit. The ethanol can be removed from the mixture by distillation and can be upgraded through rectification and de-hydration to fuel grade ethanol. The process of distillation typically thermally inactivates the yeast and enzymes that may be present in the fermentation mixture.

The bottoms, or “stillage,” from the distillation can be sent to a centrifuge or other device for separating solids from liquids, where the solid in the stillage can be separated from the liquid in the stillage. The liquid fraction can be sent to the waste treatment plant for digestion. The solids fraction can be diverted to a biomass boiler where they can be burned as fuel to produce steam, or steam and electricity, which can be used in the process.

The overhead from the distillation generally has a higher ratio of ethanol to water and the amount of ethanol can be increased by treating the overhead in a second distillation column, or rectifier, to produce an overhead that has a composition that is at or near the water-ethanol azeotrope. The bottoms from the rectifier, which can be mainly water, can be used in various process steps as set forth herein where water or a liquid stream containing water is used, such as in washing solids during solid/liquid separation steps or in the hydrolysis step. For example, at least part of the bottoms from the rectifier can be used to provide additional water for the steam explosion of the solids produced during the hydrolysis reaction. It can be used as source of heat for one or more of the process steps as set forth in the processes of this invention. Similarly, the liquid stream that is primarily water that is obtained from the stillage, either before or after treatment with, for example, waste water treatment procedures to remove toxic compounds, can also be used in one or more of the various process steps as set forth herein where water or a liquid stream containing water is used, such as in processing the feedstock to form the first juice or washing solids during solid/liquid separation step following the hydrolysis reaction, or as water for the hydrolysis step.

### Distribution and Use of the First Juice, Hydrolyzate, First Fermentation Mixture and Second Fermentation Mixture

As described above, the first juice can be a solution of a sugar, such as sucrose, in water. For the following embodiments, the first juice can, for example, be any of the embodiments of the first juice described hereinabove. At least part of the first juice can be used in the step of the process where the mixture of solids and liquids produced by the hydrolysis step are separated to produce the liquid and the solid portion. For example, at least part of the first juice can be used to wash solids to assist with the removal of any hydrolyzate adhering, entrained in or otherwise combined with the solids in the mixture of solid and liquid formed by the hydrolysis step. For example, the first juice can be used to wash the solids in the one or more separation apparatuses, such as the screw presses, used to undertake the solid-liquid separation of the liquid from the solids produced in the hydrolysis step. First juice added at this step of the process can become part of the hydrolyzate mixture subjected to detoxification and then is sent to the vessel used to conduct the fermentation of the hydrolyzate where any sugars in the first juice can be fermented along with the sugar or sugars in the hydrolyzate to form ethanol. Using the first juice that contains a sugar, such as sucrose, to wash the solids provides a wash liquid that, after fermentation, produces ethanol thereby adding to the amount of ethanol produced by the overall process. If water without a sugar content is used to wash the solids, that water would need to be removed to produce purified ethanol. In this invention, the use of a first juice containing a sugar reduces the overall amount of water that would need to be removed relative to the amount of ethanol produced compared to a process where water without a sugar is used to wash the solids produced in the hydrolysis step.

Adding the first juice comprising one or more sugars as a wash for the solid-liquid separation of the liquid from the solids produced in the hydrolysis step can assist with the fermentation of the sugars in the hydrolyzate to form ethanol. For example, there can be an improvement in the fermentation of the C5 sugars in the hydrozylate when fermented along with the sugars in the first juice. The use of a first juice as a wash for the solid-liquid separation of the liquid from the solids produced in the hydrolysis step dilutes the hydrolyzate and thereby decreases the concentration of toxins in the hydrolyzate that are produced during the hydrolysis step and such reduction in concentration of the toxins can improve the fermentation of the sugars in the diluted hydrolyzate.

Suitably, all of the first juice is used as a wash liquid to wash the solids during the step of separating the liquids and solids produced by the hydrolysis reaction. However, only about 95 to about 99, or about 90 to about 94, or about 85 to about 89, or about 80 to about 84, or about 75 to about 79, or about 70 to about 74, about 65 to about 69, or about 60 to about 64, or about 55 to about 59, or about 50 to about 54, about 45 to about 49, about 40 to about 44, about 35 to about 39, about 30 to about 34, about 25 to about 29, about 20 to about 24, about 15 to about 19, about 10 to about 14, about 5 to about 9, about 1 to about 4 volume percent of the first juice can be used as a wash liquid during the step of separating the liquids and solids produced by the hydrolysis reaction.

At least part of the first juice can be added to the solid portion recovered from the hydrolysis either prior to or during the saccharification and fermentation of the solids portion. For example, at least part of the first juice can be added to the solid portion after the solid portion exits the separation apparatus or apparatuses used to separate the mixture of solids and liquids produced by the hydrolysis step. At least part of the first juice can be added to a vessel along with that solid portion to form slurry comprising the solid portion and at least part of the first juice. This slurry can be added to the vessel used to perform the saccharification and fermentation of the solids portion. At least part of the first juice can be added to the vessel used to perform the saccharification and fermentation of the solids portion.

At least part of the first juice can be used to prepare microorganisms, such as one or more yeasts, that are, for example, used in the disclosed processes for the fermentation of the hydrolyzate, for example, the fermentation of the C5 sugars, and/or the fermentation of the sugars produces by the saccharification of the solids portion produced by the hydrolysis as described herein.

At least part of the first juice can be used in processes, such as biological processes, of converting a sugar to one or more other alcohols, such as one or more butanols, for example, isobutanol. At least part of the first juice can be used in other process for the conversion of sugars by biological processes, for example, known biological processes, to triglycerides that can be used, for example, for biodiesel fuels. At least part of the first juice can be used in processes, such as biological process, for example, known biological processes, for the conversion of the sugar into one or more chemical compounds such as carboxylic acids, for example, succinic, malic, and lactic acid. At least part of the first juice can be used in processes, such as biological process, for example, known biological processes, for the production of enzymes, for examples, enzymes used for the saccharification of cellulose into glucose.

At least part of a first juice comprising one or more sugars can be fermented in, for example, a fermentation vessel, to form a fermented first juice. The fermentation of sugars in a first juice can be partial or total. Any suitable method for fermenting such a first juice can be used. Before being used as described below, the fermented first juice can be treated to remove any microorganisms and/or other insoluble materials that may be present in the fermented first juice. For example, it can be filtered, centrifuged, treated in a cyclone, subject to settling, and the like to remove any microorganisms and/or other insoluble material in the fermented first juice.

This fermented first juice can be used in the step of the process where the mixture of solids and liquids produced by the hydrolysis step are separated to produce the liquid and the solid portion. For example, at least part of the fermented first juice can be used to wash solids to assist with the removal of any hydrolyzate adhering, entrained in or otherwise combined with the solids in the mixture of solid and liquid formed by the hydrolysis step. For example, at least part of the fermented first juice can be used to wash the solids in the one or more apparatuses used to undertake the solid-liquid separation of the liquid from the solids produced in the hydrolysis step.

At least part of the fermented first juice can be added to the solid portion recovered from the hydrolysis either prior to or during the saccharification and fermentation of the solids portion. At least part of the fermented first juice can be added to the solid portion after the solid portion exits the separation apparatus or apparatuses used to separate the mixture of solids and liquids produced by the hydrolysis step. At least part of the first juice can be added to a vessel along with the solid portion to form a slurry comprising the solid portion and at least part of the first juice. This slurry can be added to the vessel used to perform the saccharification and fermentation of the solids portion. At least part of the fermented first juice can be added to the vessel used to perform the saccharification and fermentation of the solids portion.

At least part of the fermented first juice can be used as a wash liquid during the processing of the feedstock. For example, at least part of the fermented first juice can be added in conjunction with or as a replacement for any additional water used to remove additional sugar or sugars such as sucrose from the feedstock. The fermented first juice can be added to the solids between a first and second press or at a second press, between a second and third press, or at a third press, or at any or all of these locations.

At least part of the fermented first juice can be added to the hydrolysis step. This amount of fermented first juice added can be in conjunction with or in place of water that is suitably otherwise added to the hydrolysis step.

At least part of the first fermentation mixture can be used in the step of the process where the mixture of solids and liquids produced by the hydrolysis step are separated to produce the liquid and the solid portion. For example, at least part of the first fermentation mixture can be used to wash solids to assist with the removal of any hydrolyzate adhering, entrained in or otherwise combined with the solids in the mixture of solid and liquid formed by the hydrolysis step. For example, at least part of the first fermentation mixture can be used to wash the solids in the one or more apparatuses used to undertake the solid-liquid separation of the liquid from the solids produced in the hydrolysis step.

At least part of the first fermentation mixture can be added to the solid portion recovered from the hydrolysis prior to or during the saccharification and fermentation of the solids portion. For example, at least part the first fermentation mixture can be added to the solid portion after the solid portion exits the separation apparatus or apparatuses used to separate the mixture of solids and liquids produced by the hydrolysis step. Addition of the first fermentation mixture reduces the viscosity of the solids and increases solids pumpability, that is, the mixture of solids and first fermented mixture can be pumped with less mechanical effort. At least part of the first fermentation mixture can be added to a vessel along with the solid portion to form a slurry comprising the solid portion and at least part of the first fermentation mixture. For example, about 95 to about 99, or about 90 to about 94, or about 85 to about 89, or about 80 to about 84, or about 75 to about 79, or about 70 to about 74, about 65 to about 69, or about 60 to about 64, or about 55 to about 59, or about 50 to about 54, about 45 to about 49, about 40 to about 44, about 35 to about 39, about 30 to about 34, about 25 to about 29, about 20 to about 24, about 15 to about 19, about 10 to about 14, about 5 to about 9, about 1 to about 4 volume percent of the first fermentation mixture can be added to a vessel along with the solid portion to form a slurry comprising the solid portion and the first fermentation mixture. This slurry can be added to the vessel used to perform the saccharification and fermentation of the solids portion. At least part of the first fermentation mixture can be added to the vessel used to perform the saccharification and fermentation of the solids portion.

At least part of the first fermentation mixture can be used as a wash liquid during the processing of the feedstock. For example, at least part of the first fermentation mixture can be added in conjunction with or as a replacement for additional water used to remove additional sugar or sugars such as sucrose from the feedstock. At least part of the first fermentation mixture can be added to the solids between a first and second press or at a second press, between a second and a third press, or at a third press, or at any or all of these locations.

At least part of the first fermentation mixture can be added to the hydrolysis step. This amount of first fermented juice added can be used with or in place of water that is suitably otherwise added to the hydrolysis step.

Part of the second fermentation mixture can be used in the step of the process where the mixture of solids and liquids produced by the hydrolysis step are separated to produce the solid portion and the liquid. For example, part of the second fermentation mixture can be used to wash solids to assist with the removal of any hydrolyzate adhering, entrained in or otherwise combined with the solids in the mixture of solid and liquid formed by the hydrolysis step. For example, part of the second fermentation mixture can be used to wash the solids in the one or more apparatuses used to undertake the solid-liquid separation of the liquid from the solids produced in the hydrolysis step.

Part of the second fermentation mixture can be added to the solid portion recovered from the hydrolysis prior to or during the saccharification of the solids portion. For example, part of the second fermentation mixture can be added to the solid portion after the solid portion exits the separation apparatus or apparatuses used to separate the mixture of solids and liquids produced by the hydrolysis step. Part of the second fermentation mixture can be added to a vessel along with the solid portion to form a slurry comprising the solid portion. This slurry can be added to the vessel used to perform the saccharification of the solids portion.

Part of the second fermentation mixture can be used as a wash liquid during the processing of the feedstock. For example, part of the second fermentation mixture can be added in conjunction with or as a replacement for the additional water used to remove additional sugar or sugars such as sucrose during the processing of the feedstock. Part of the second fermentation mixture can be added to the solids between a first and second press or at a second press, between a second and third press, or at a third press, or at any or all of these locations, where these presses are used to press the first juice from the feedstock.

Part of the second fermentation mixture can be added to the hydrolysis step. This amount of the second fermentation mixture added can be used with or in place of water that is suitably otherwise added to the hydrolysis step.

At least part of the hydrolysate, either before detoxification or after detoxification, can be used in processes, such as biological processes, of converting a sugar to one or more other alcohols, such as one or more butanols, for example, isobutanol. At least part of such hydrolysate can be used in other process for the conversion of sugars by biological processes, for example, known biological processes, to triglycerides that can be used, for example, for biodiesel fuels. At least part of such hydrolyzate can be used in processes, such as biological process, for example, known biological processes, for the conversion of the sugar into one or more chemical compounds such as carboxylic acids, for example, succinic, malic, and lactic acid. At least part of such hydrolyzate can be used in processes, such as biological process, for example, known biological processes, for the production of enzymes, for examples, enzymes used for the saccharification of cellulose into glucose.

Before using the hydrolyzate as set forth above, either before or after a detoxification treatment, it can be treated to remove insoluble materials that may be present. For example, it can be filtered, centrifuged, treated in a cyclone, subject to settling, and the like to remove any insoluble materials.

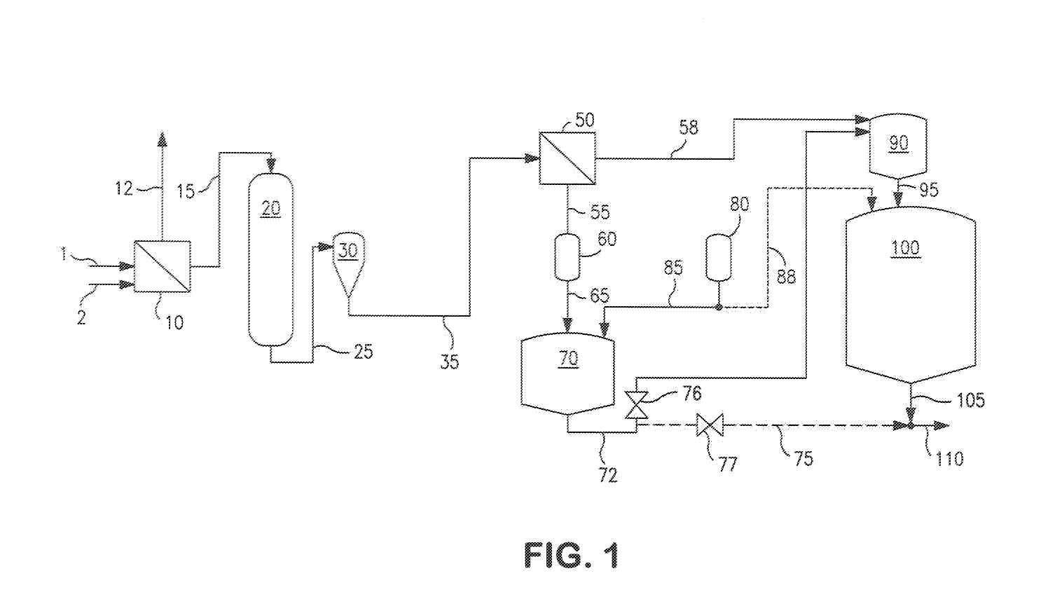

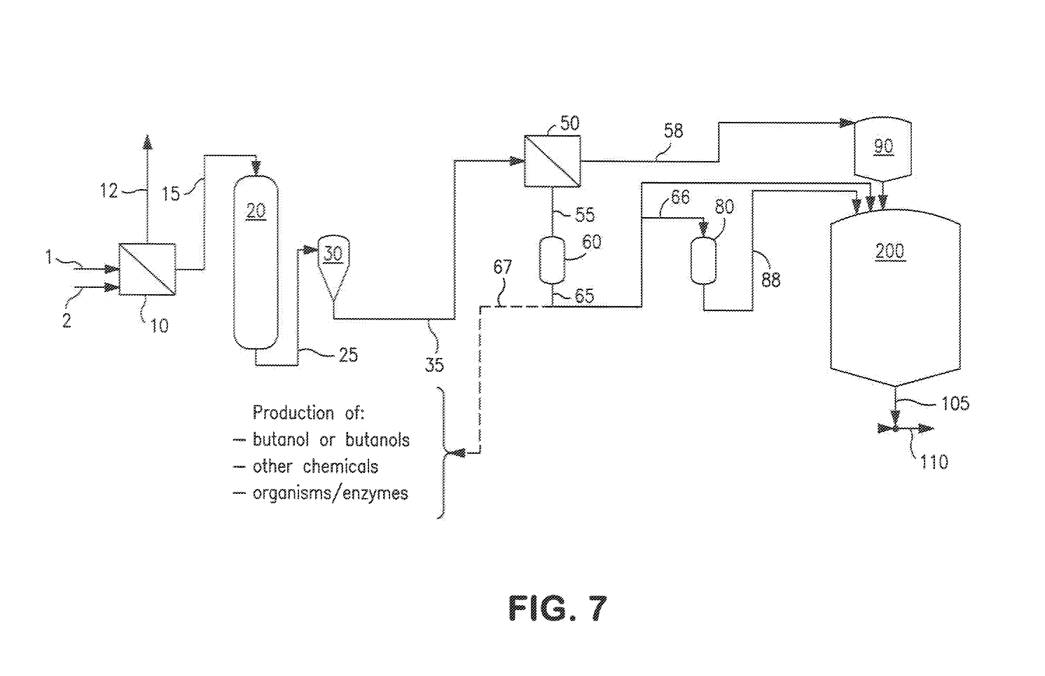

Embodiments of the invention will now be described with respect to the accompanying FIGS. 1 through 8. These figures are schematic representations of embodiments of the processes disclosed herein. The various apparatus depicted in these figures are not necessarily drawn to scale. Elements in each drawing that are numbered the same refer to the same element.

FIG. 1 is a process flow diagram showing a process for the conversion of a feedstock to ethanol in accordance with an embodiment of this invention.

As shown in FIG. 1, comminuted feedstock enters through line 1 a series of three roller presses 10 where the comminuted feedstock is treated to form the first juice which exits roller presses 10 through line 12 and solids which exit roller presses 10 through line 15. Water for washing the solids produced by the roller presses is supplied to the presses through line 2. Solids enter hydrolysis reaction vessel 20 where they are subjected to dilute acid hydrolysis at elevated temperature and pressure. Hydrolysis reaction mixture under pressure exits hydrolysis reaction vessel 20 through line 25 and enters blow cyclone 30 where it is rapidly depressurized and undergoes “steam explosion” to further reduce the particle size and disrupt the structure of the cellulosic materials contained therein. The material produced in the steam explosion can be held in a an appropriate vessel. The mixture of liquid and solids produced by the hydrolysis reaction and subsequent steam explosion step exits cyclone 30 through line 35 and enters screw presses 50 where the liquid in the mixture of liquid and solids produced by the hydrolysis reaction and subsequent steam explosion step is separated from the solids to form the liquid hydrolyzate and the solids portion comprising the cellulosic material that will later be subjected to saccharification to form glucose. Hydrolyzate exits screw presses 50 through line 55 and enters reaction vessels 60 (for simplicity, only one reaction vessel is depicted in the figures) where the hydrolyzate is subjected to detoxification in detoxification vessel 60. Detoxified hydrolyzate exits detoxification vessel 60 through line 65 and enters first fermentation vessels 70 (for simplicity, only one vessel depicted in the figures) where the sugars, for example, xylose, in the detoxified hydrolyzate are fermented to form first fermentation mixture comprising ethanol. First fermentation vessel 70 can be operated in the fed-batch mode whereby the materials used for the fermentation are added to the suitably stirred fermentation vessel while the fermentation is proceeding within the vessel. After the vessel is filled to the desired level, the fermentation is allowed to proceed further until, for example, the desired conversion of the sugars in the hydrolyzate, for example, xylose, to ethanol is achieved. The selected microorganism or microorganisms for the fermentation of the hydrolyzate and for the fermentation of glucose are stored in storage vessels 80 (for simplicity, only one storage vessel is shown in the figures). The selected microorganism for the fermentation of the hydrolyzate exits storage vessel 80 through line 85 and enters first fermentation vessel 70. A portion of the first fermentation mixture exits the fermentation vessel 70 through line 72 and enters slurry vessel 90 as will be described in more detail below. Valve 76 can be used to regulate the amount and rate of flow of the first fermentation mixture through line 72 and consequently the amount of first fermentation mixture that is sent to slurry vessel 90. Solids portion comprising the cellulosic material exits screw presses 50 through line 58 and enters slurry vessel 90 where it is suitably mixed with first fermentation mixture from fermentation vessel 70 to form a slurry of the solids portion and the first fermentation mixture. A suitable base, such as ammonia or magnesium hydroxide, can be added to slurry vessel 90 to, for example, neutralize any acidic components that may remain with the solids portion comprising the cellulosic material. The slurry exits slurry vessel 90 through line 95 and enters saccharification and fermentation vessel 100 where the cellulosic material in the solids portion is saccharified and the resulting glucose from the saccharification is simultaneously fermented to ethanol in second fermentation mixture in saccharification and fermentation vessel 100. The fermentation in saccharification and fermentation vessel 100 can be a fed-batch saccharification and fermentation. Suitable microorganisms for the fermentation in the saccharification and fermentation vessel 100, such as a yeast, can be supplied to the saccharification and fermentation vessel 100 from storage vessels 80 through line 88. Line 88 is shown as a dotted line in FIG. 1 because it is an optional supply line. Otherwise, for example, fermentation microorganisms can be supplied to saccharification and fermentation vessel 100 as part of the first fermentation mixture supplied to slurry tank 90 and then into saccharification and fermentation vessel 100 through line 95 along with the slurry from slurry vessel 90. The slurry in slurry vessel 90 is added to vessel 200 along with suitable enzymes or an enzyme mixture and optionally with additional microorganisms such as a yeast, to undertake the saccharification and fermentation of the solids in vessel 200 to form a second fermentation mixture. Second fermentation mixture exits saccharification and fermentation vessel 100 through line 105 where it can be sent through line 110 to further processing, not shown in FIG. 1, to obtain purified ethanol. Valve 77 can be used in conjunction with valve 76 to regulate the amount and rate of flow of the first fermentation mixture through line 75 and consequently the amount of first fermentation mixture that is sent to slurry vessel 90. First fermentation mixture that is not sent to slurry vessel 90 can be sent to line 110 through line 75. If valve 76 is closed and valve 77 is open, for example, the process becomes a “two vessel process” where one first fermentation vessel (or collection of first fermentation vessels) 70 is used for the fermentation of the hydrolyzate and one saccharification and fermentation vessel (or collection of saccharification and fermentation vessels) 100 is used for the saccharification and fermentation of the cellulosic material.

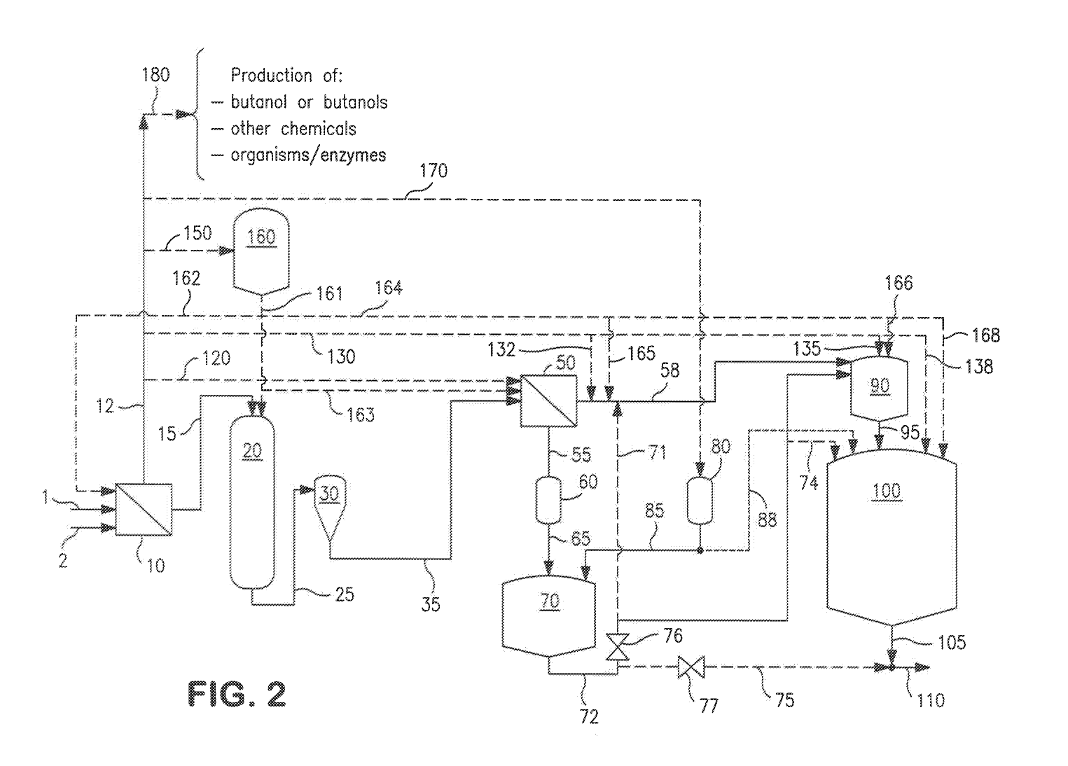

FIG. 2 is a process flow diagram showing a process for the conversion of a feedstock to ethanol in accordance with embodiments of this invention where the first juice stream is used in various process steps.

A shown in FIG. 2, first juice stream 12 that exits roller presses 10 can be used in various process steps for the production of ethanol in accordance with embodiments of the invention. In FIG. 2 the dotted lines mean that these process flows in particular can be run with variability. For example, at any time during the manufacturing process, all can be run to some extent or only one or more.

As shown in FIG. 2, at least part of first juice stream can exit line 12 and be sent through line 120 to screw presses 50 to, for example, wash the solids produced in the screw presses. At least part of first juice stream can exit line 12 and be sent through lines 130 and 132 to be added to the solid portion after the solid portion exits the screw presses 50. The first juice used in this manner improves, for example, the pumpability of the solids portion. At least part of first juice stream can exit line 12 and be sent through lines 130 and 135 to be added to slurry vessel 90 where it is mixed with the solids portion recovered from the hydrolysis step of the process. At least part of first juice stream can exit line 12 and be sent through lines 130 and 138 to saccharification and fermentation vessel 100 where it is mixed with the solids portion recovered from the hydrolysis step of the process and the sugars contained within the first juice would be co-fermented with the sugars produced by the saccharification process within saccharification and fermentation vessel 100.

As shown in FIG. 2, at least part of first juice stream 12 can be sent through line 150 to a second fermentation vessel 160 where any sugars contained in the first juice stream can be totally or partially fermented to produce a fermented first juice. This fermented first juice can, in accordance with embodiments of the invention can, as will be discussed below, be used in various process steps.

As shown in FIG. 2, at least part of first juice stream 12 can be sent through line 170 to storage vessel (or storage vessels) 80 where it can be used to prepare microorganisms, such as one or more yeasts, that are used in the disclosed processes for the fermentation of the hydrolyzate, for example, the C5 sugars, and/or the fermentation in the SSF step of the disclosed process.

As shown in FIG. 2, at least part of the first juice stream 12 can be sent through line 180 to be used in one or more processes, such as biological processes, for converting a sugar to one or more other alcohols, such as one or more butanols, for example, isobutanol; or can be used in processes for the conversion of sugars by biological processes, for example, known biological processes, to triglycerides that can be used, for example, for biodiesel fuels; or can be used in processes, such as biological process, for example, known biological processes, for the conversion of the sugar into one or more chemical compounds such as carboxylic acids, for example, succinic, malic, and lactic acid; or can be used in processes, such as biological process, for example, known biological processes, for the production of enzymes, for examples, enzymes used for the saccharification of cellulose to form glucose.

As shown in FIG. 2, at least part of the fermented first juice produced in second fermentation vessel 160 can be sent through lines 161 and 162 to roller presses 10 to be used, for example, as a wash liquid during the processing of the feedstock. For example, the fermented first juice can be added either in conjunction with or as a replacement for the additional water used to remove additional sugar or sugars such as sucrose from the feedstock.

As shown in FIG. 2, at least part of fermented first juice can be sent through lines 161, 164 and 165 to be added to the solid portion after the solid portion exits the screw presses 50. The fermented first juice used in this manner improves, for example, the pumpability of the solids portion. At least part of the fermented first juice can be sent through lines 161, 164 and 166 to be added to slurry vessel 90 where it is mixed with the solids portion recovered from the hydrolysis step of the process. The viscosity of the contents of the fermentation and saccharification vessel 100 can be reduced by the addition of such process stream making it easier to stir that mixture and thereby promoting the saccharification and fermentation processes. At least part of fermented first juice can be sent through lines 161, 164 and 168 to fermentation and saccharification vessel 100 where it is mixed with the solids portion recovered from the hydrolysis step of the process.

As shown in FIG. 2, at least part of the fermented first juice can be sent through line 161 to hydrolysis reaction vessel 20 to be added to the hydrolysis step. This amount of fermented first juice added can be used with or in place of water that is suitably otherwise added to the hydrolysis step. As shown in FIG. 2, at least part of fermented first juice stream can exit line 161 and be sent through line 163 to screw presses 50 to, for example, wash the solids produced in the screw presses.

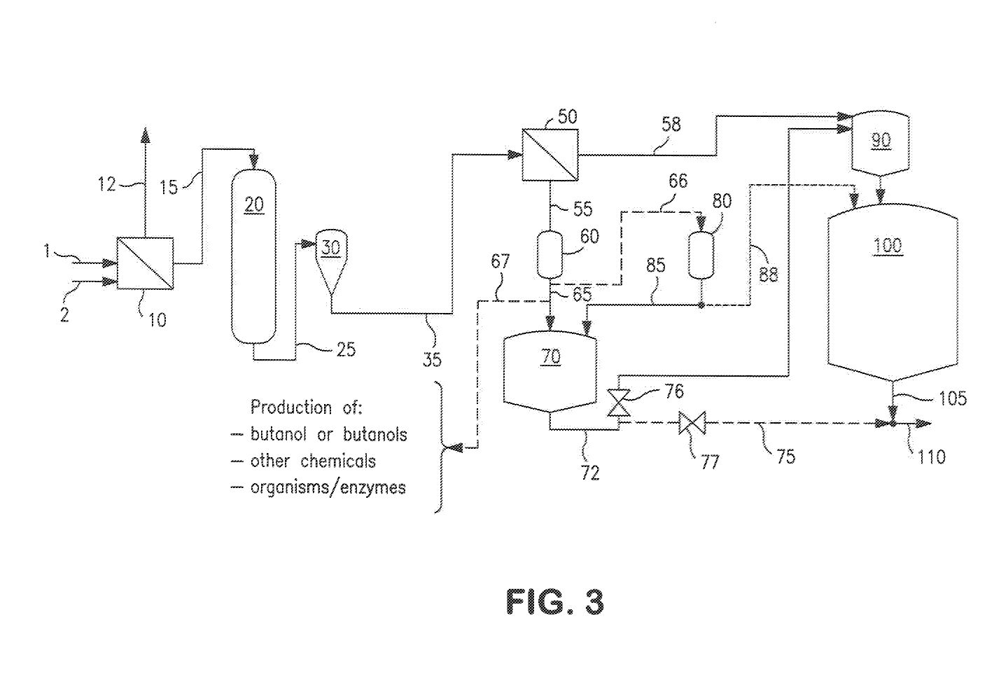

FIG. 3 is a process flow diagram showing a process for the conversion of a feedstock to ethanol in accordance with embodiments of this invention where the hydrolyzate stream is used in various process steps.

As shown in FIG. 3, part at least part of the hydrolyzate exiting detoxification vessel 60 can be sent through lines 65 and 66 to vessel 80 (or vessels 80) where it can be used to prepare microorganisms, such as one or more yeasts, that are used in the disclosed processes for the fermentation of the hydrolyzate, for example, the C5 sugars, and/or the fermentation in the SSF step of the disclosed process. At least part of the hydrolyzate exiting detoxification vessel 60 can be sent through lines 65 and 67 where it can be used in processes, such as biological processes, for converting a sugar to one or more other alcohols, such as one or more butanols, for example, isobutanol; or where at least part of it can be used in other process for the conversion of sugars by biological processes, for example, known biological processes, to triglycerides that can be used, for example, for biodiesel fuels; or where at least part of it can be used in processes, such as biological process, for example, known biological processes, for the conversion of the sugars into one or more chemical compounds such as carboxylic acids, for example, succinic, malic, and lactic acid; or where at least part of it can be used in processes, such as biological process, for example, known biological processes, for the production of enzymes, for example, enzymes used for the saccharification of cellulose into glucose.

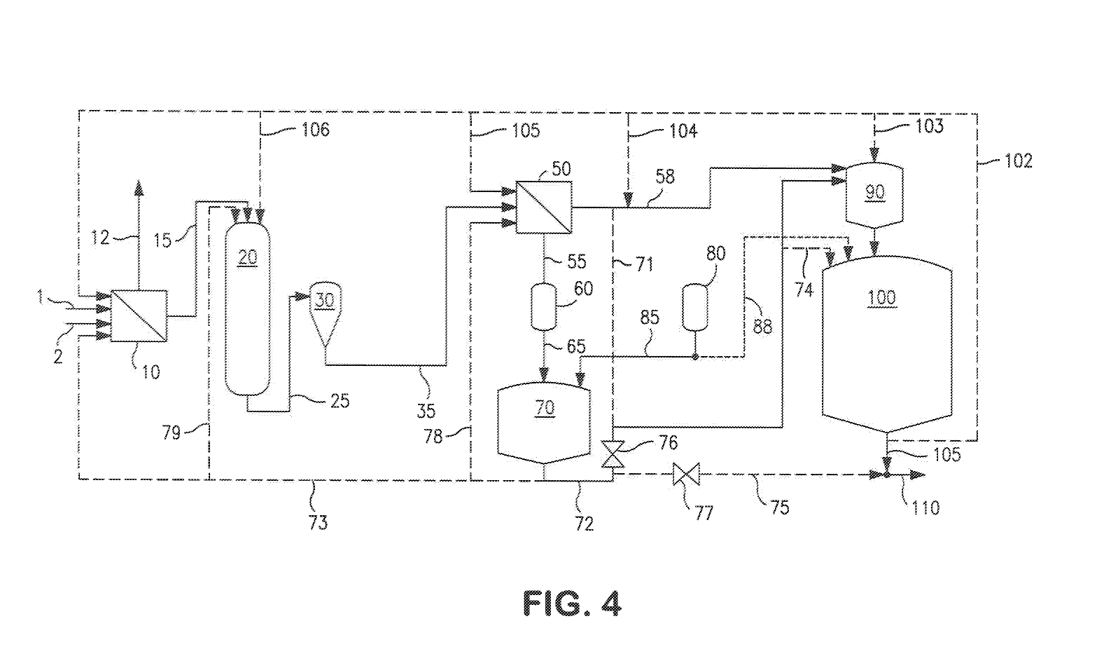

FIG. 4 is a process flow diagram showing a process for the conversion of a feedstock to ethanol in accordance with embodiments of this invention.

As shown in FIG. 4, at least part of the first fermentation mixture produced in first fermentation vessel 70 can be sent through lines 73 to roller presses 10 to be used, for example, as a wash liquid during the preparation of the feedstock. For example, the first fermentation mixture can be added either in conjunction with or as a replacement for the additional water used to remove additional sugar or sugars such as sucrose from the feedstock.

As shown in FIG. 4, at least part of first fermentation mixture can be sent through lines 71 and 72 to be added to the solids portion after the solids portion exits the screw presses 50. The first fermentation mixture used in this manner improves, for example, the pumpability of the solids portion. At least part of the first fermentation mixture can be sent through line 72 to be added to slurry vessel 90 where it is mixed with the solids portion recovered from the hydrolysis step of the process. The viscosity of the contents of the fermentation and saccharification vessel 100 can be reduced by the addition of such process stream making it easier to stir that mixture and thereby promoting the saccharification and fermentation processes. At least part of first fermentation mixture can be sent through lines 72 and 74 to fermentation and saccharification vessel 100 where it is mixed with the solids portion recovered from the hydrolysis step of the process.

As shown in FIG. 4, at least part of the first fermentation mixture can be sent through lines 73 and 79 to hydrolysis reaction vessel 20 to be added to the hydrolysis step. This amount of first fermentation mixture added can be used with or in place of water that is suitably otherwise added to the hydrolysis step. As shown in FIG. 4, at least part of first fermentation mixture can be sent through line 73 and 78 to screw presses 50 to, for example, wash the solids produced in the screw presses.

As shown in FIG. 4, part of the second fermentation mixture produced in fermentation and saccharification vessel 100 can be sent through line 102 to roller presses 10 to be used, for example, as a wash liquid during the processing of the feedstock. For example, the second fermentation mixture can be added either in conjunction with or as a replacement for the additional water used to remove additional sugar or sugars such as sucrose from the feedstock.

Part of the second fermentation mixture can be sent through lines 102 and 103 to be added to slurry vessel 90 where it is mixed with the solids portion recovered from the hydrolysis step of the process. The viscosity of the contents of the slurry vessel 90 can be reduced by the addition of such process stream making it easier to stir that mixture. As shown in FIG. 4, part of second fermentation mixture can be sent through lines 102 and 104 to be added to the solids portion after the solids portion exits the screw presses 50. The second fermentation mixture used in this manner improves, for example, the pumpability of the solids portion. As shown in FIG. 4, part of second fermentation mixture can be sent through line 102 and 105 to screw presses 50 to, for example, wash the solids produced in the screw presses. Part of second fermentation mixture can be sent through lines 102 and 106 to hydrolysis reaction vessel 20 to be added to the hydrolysis step. This amount of second fermentation mixture added can be used with or in place of water that is suitably otherwise added to the hydrolysis step.

Although as shown in FIGS. 1 through 4 fermentation of the hydrolyzate can take place in first fermentation vessel 70, embodiments of this invention include conducting the fermentation of the hydrolyzate in the same vessel used for the saccharification of the solids recovered from the hydrolysis of the feedstock and subsequent fermentation of the sugars produced by the saccharification. In this embodiment, the hydrolyzate is sent to a vessel or vessels used for the saccharification and fermentation of the solids portion separated after the hydrolysis step, the hydrolyzate is fermented either partially or totally in that vessel to form the first fermentation mixture and then the solids portion is added to the same vessel along with the appropriate enzymes to undertake the saccharification and fermentation of the solids.

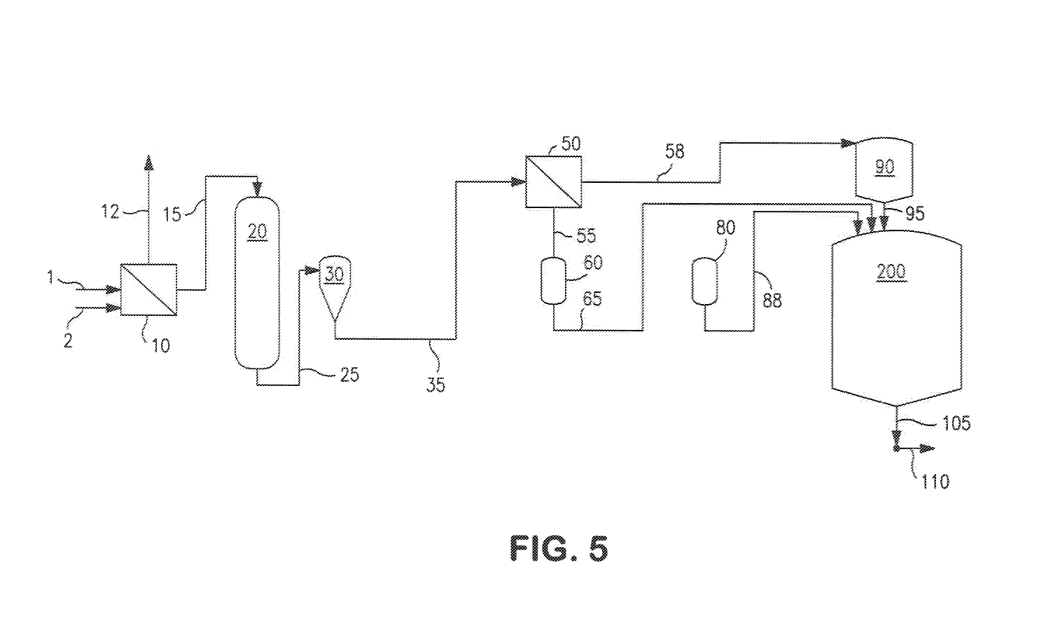

FIGS. 5 through 8 show process flow diagrams for such processes.

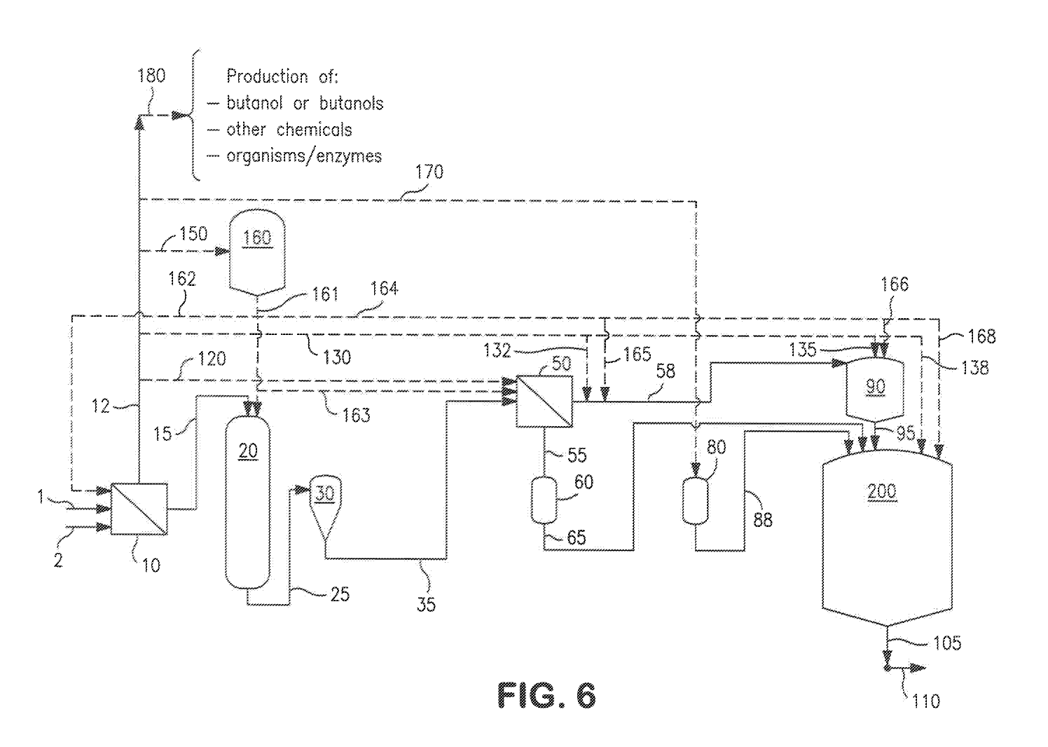

As shown in FIGS. 5, 6 7 and 8, hydrolyzate exits detoxification vessel 60 and is sent through line 65 to vessel 200. A suitable microorganism for fermenting sugars in the hydrolyszate can be added from vessel 80 to vessel 200 through line 88. The hydrolyzate is totally or partially fermented in vessel 200 to form the first fermentation mixture. The solids from screw presses 50 are sent through line 58 and into slurry vessel 90 where they can be mixed with one or more suitable liquids to form a slurry and/or subjected to partial saccharification. The partial saccharification can reduce the viscosity of the solid in the slurry vessel. The slurry in slurry vessel 90 is added to vessel 200 along with suitable enzymes or an enzyme mixture and optionally with additional microorganisms such as a yeast, to undertake the saccharification and fermentation of the solids in vessel 200 to form a second fermentation mixture.

The other process streams shown in FIGS. 5-8 are the same as those shown in FIGS. 1-4.

For example, as shown in FIG. 6, at least part of first juice stream can exit line 12 and be sent through line 120 to screw presses 50 to, for example, wash the solids produced in the screw presses. At least part of first juice stream can exit line 12 and be sent through lines 130 and 132 to be added to the solids portion after the solids portion exits the screw presses 50. The first juice used in this manner improves, for example, the pumpability of the solids portion. At least part of first juice stream can exit line 12 and be sent through lines 130 and 135 to be added to slurry vessel 90 where it is mixed with the solids portion recovered from the hydrolysis step of the process. At least part of first juice stream can exit line 12 and be sent through lines 130 and 138 to combined first fermentation and saccharification and fermentation vessel 200. Sugars in the first juice stream can be co-fermented with the sugars produced by the saccharification process within vessel 200.

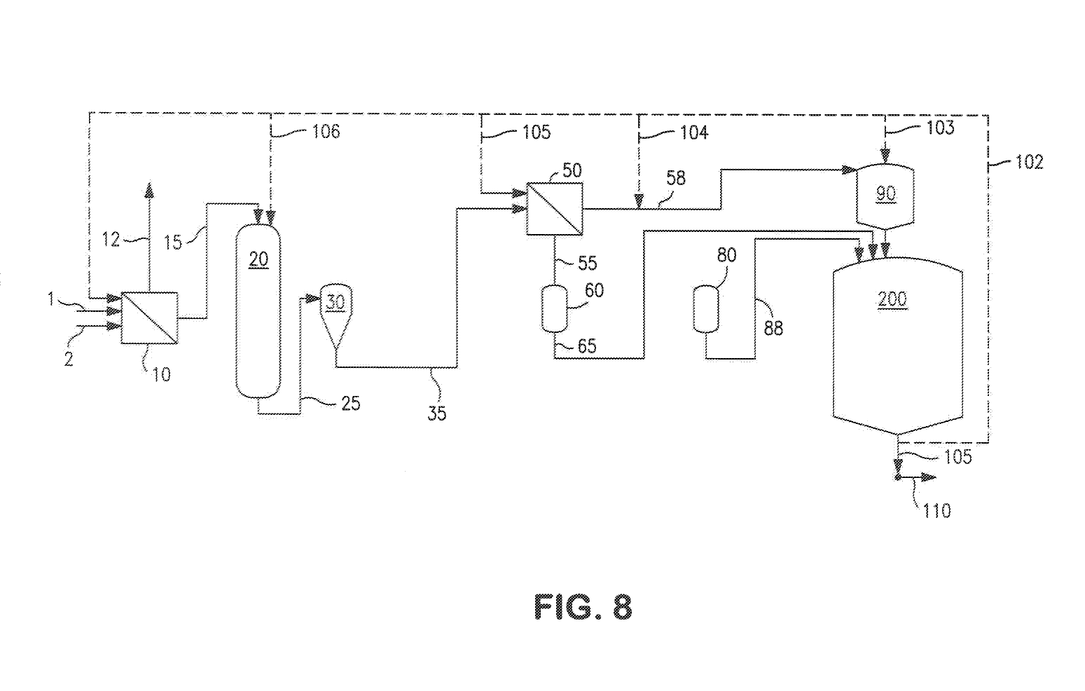

As shown in FIG. 8, at least part of the first fermentation mixture and/or part of the second fermentation mixture produced in vessel 200 can be sent through line 102 to roller presses 10 to be used, for example, as a wash liquid during the preparation of the feedstock. For example, at least part of the first fermentation mixture and/or part of the second fermentation mixture can be added either in conjunction with or as a replacement for the additional water used to remove additional sugar or sugars such as sucrose from the feedstock.

As shown in FIG. 8, at least part of the first fermentation mixture and/or part of the second fermentation mixture can be sent through lines 102 and 103 to be added to slurry vessel 90 where it is mixed with the solids portion recovered from the hydrolysis step of the process. The viscosity of the contents of the slurry vessel 90 can be reduced by the addition of such process stream making it easier to stir that mixture.

As shown in FIG. 8, at least part of the first fermentation mixture and/or part of the second fermentation mixture can be sent through lines 102 and 104 to be added to the solids portion after the solids portion exits the screw presses 50. A fermentation mixture used in this manner improves, for example, the pumpability of the solids portion. As shown in FIG. 8, at least part of the first fermentation mixture and/or part of the second fermentation mixture can be sent through line 102 and 105 to screw presses 50 to, for example, wash the solids produced in the screw presses. At least part of the first fermentation mixture and/or part of the second fermentation mixture can be sent through lines 102 and 106 hydrolysis reaction vessel 20 to be added to the hydrolysis step. This amount of fermentation mixture added can be used with or in place of water that is suitably otherwise added to the hydrolysis step.

It will be apparent to those skilled in the art that various modifications and variations can be made in the disclosed structures processes and methods without departing from the scope or spirit of the invention. Other embodiments of the invention will be apparent to those skilled in the art from consideration of the specification and practice of the invention disclosed herein. It is intended that the specification be considered exemplary only, with a true scope and spirit of the invention being indicated by the following claims.

That which is claimed is:

1. A process for converting a cellulosic feedstock to one or more chemical compounds, comprising:
a) processing the feedstock to form a first juice and a first solids comprising cellulose and hemicellulose;
b) hydrolyzing at least part of the first solids to form a first liquid comprising one or more sugars and second solids comprising cellulose;
c) separating in one or more separation apparatuses first liquid from second solids to form a first liquid portion comprising one or more sugars and a solids portion comprising cellulose;
d) fermenting at least part of the one or more sugars in the first liquid portion to form a first fermentation mixture comprising one or more chemical compounds;
e) saccharifying in the presence of at least a portion of the first fermentation mixture at least part of the solids portion to form a second mixture comprising one or more sugars;
f) fermenting at least a portion of the sugars in the second mixture to form a second fermentation mixture comprising one or more chemical compounds; and
at least one of the following additional steps:
i) washing second solids with at least part of the first juice;
ii) adding at least a part of the first juice to solids portion exiting the one or more separation apparatuses;
iii) combining in a slurry vessel at least a part of the first juice with at least a part of the solids portion comprising cellulose;
iv) saccharifying solids portion in the presence of at least part of the first juice; and
v) and fermenting sugars in the second mixture in the presence of at least part of the first juice.

2. The process of claim 1 wherein the first juice comprises one or more sugars.

3. The process of claim 1 wherein the processing comprises pressing the feedstock to form the first juice.

4. The process of claim 1 wherein the first juice comprises sucrose.

5. The process of claim 1 comprising step i) and wherein at least part of the first juice used for the washing is combined with first liquid.

6. The process of claim 1 wherein at least part of the saccharifying in step e) and at least part of the fermentation in step f) occur simultaneously.

7. The process of claim 1 wherein the additional steps is i) and all or substantially all of the first juice is used in washing second solids.

8. The process claim 1 wherein the washing is in one or more separation apparatuses.

9. The process claim 1 wherein step e) is subsequent to step d).

10. A process for converting a cellulosic feedstock to one or more chemical compounds, comprising:
a) processing the feedstock to form a first juice and a first solids comprising cellulose and hemicellulose;
b) hydrolyzing at least part of the first solids to form a first liquid comprising one or more sugars and second solids comprising cellulose;
c) separating in one or more separation apparatuses first liquid from second solids to form a first liquid portion comprising one or more sugars and a solids portion comprising cellulose;
d) fermenting at least part of the one or more sugars in the first liquid portion to form a first fermentation mixture comprising one or more chemical compounds;
e) saccharifying at least part of the solids portion to form a second mixture comprising one or more sugars;
f) fermenting at least a portion of the sugars in the second mixture to form a second fermentation mixture comprising one or more chemical compounds; and
at least one of the following additional steps:
i) washing in the separation apparatus second solids with at least part of the first fermentation mixture;
ii) adding at least a part of the first fermentation mixture to solids portion exiting the one or more separation apparatuses;
iii) combining in a slurry vessel at least part of the first fermentation mixture with at least part of the solids portion comprising cellulose;
iv) saccharifying solids portion in the presence of at least part of the first fermentation mixture;
v) fermenting sugars in the second mixture in the presence of at least part of the first fermentation mixture;
vi) processing the feedstock with at least part of the first fermentation mixture; and
vii) hydrolyzing at least part of the first solids with at least part of the first fermentation mixture.

11. The process of claim 10 wherein the first juice comprises one or more sugars.

12. The process of claim 10 wherein the processing comprises pressing the feedstock to form the first juice and first solids, and washing the first solids with at least part of the first fermentation mixture.

13. The process of claim 10 comprising step ii) wherein the first fermentation mixture is added to the solids portion as the solids portion exits the one or more separation apparatuses and wherein the first fermentation mixture reduces the viscosity of the solids portion.

14. The process of claim 10 comprising step iii) wherein the first fermentation mixture reduces the viscosity of the solids portion.

15. The process of claim 10 wherein the additional step is iii).

16. The process of claim 15 wherein all or substantially all of the first fermentation mixture is combined with the solids portion comprising cellulose.

17. The process of claim 10 wherein at least part of the saccharifying in step e) and at least part of the fermentation in step f) occur simultaneously.

18. The process of claim 10 wherein the saccharifying is in the presence of at least part of the first fermentation mixture.

19. The process of claim 18 wherein step e) is subsequent to step d).

20. A process for converting a cellulosic feedstock to one or more chemical compounds, comprising:
a) processing the feedstock to form a first juice and a first solids comprising cellulose and hemicellulose;
b) hydrolyzing at least part of the first solids to form a first liquid comprising one or more sugars and second solids comprising cellulose;
c) separating in one or more separation apparatuses first liquid from second solids to form a first liquid portion comprising one or more sugars and a solids portion comprising cellulose;
d) fermenting at least part of the one or more sugars in the first liquid portion to form a first fermentation mixture comprising one or more chemical compounds;
e) saccharifying, in the presence of at least part of the first fermentation mixture, at least part of the solids portion to form a second mixture comprising one or more sugars;
f) fermenting at least a portion of the sugars in the second mixture to form a second fermentation mixture comprising one or more chemical compounds; and
at least one of the following additional steps:
i) washing in the separation apparatus second solids with part of the second fermentation mixture;
ii) adding part of the second fermentation mixture to solids portion exiting the one or more separation apparatuses;
iii) combining in a slurry vessel a part of the second fermentation mixture with at least a part of the solids portion comprising cellulose;
iv) processing the feedstock with part of the second fermentation mixture; and
vi) hydrolyzing at least part of the first solids with part of the second fermentation mixture.

21. The process of claim 20 wherein the first juice comprises one or more sugars.

22. The process of claim 20 wherein the processing comprises pressing the feedstock to form the first juice and first solids, and washing first solids with part of the second fermentation mixture.

23. The process claim 20 wherein at least part of the saccharifying in step e) and at least part of the fermentation in step f) occur simultaneously.

24. The process of any one of claims 30 through 33 wherein the saccharifying is in the presence of at least a portion of the first fermentation mixture.

25. The process of claim 24 wherein step e) is subsequent to step d).

26. The process claim 1 wherein the one or more chemical compounds is ethanol.
